(19) United States
(12) Patent Application Publication
(10) Pub. No.: US 2010/0173406 A1
(43) Pub. Date: Jul. 8, 2010

(54) VIRUS MEDIATED CYTOPLASMIC EXPRESSION OF DNA VACCINES

(76) Inventor: Jacques Perrault, Poway, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/447,242

(22) PCT Filed: Nov. 7, 2007
(86) PCT No.: PCT/US07/83929

§ 371 Date: Jan. 13, 2010

Related U.S. Application Data
(60) Provisional application No. 60/857,663, filed on Nov. 7, 2006.

(51) Int. Cl. C12N 5/071; C12N 15/74

(52) U.S. Cl. 435/325; 435/320.1

(57) ABSTRACT

The present invention relates generally to methods, systems, and compositions for cytoplasmic delivery and expression of DNA vaccines. The present invention further relates to methods, systems, and compositions for expressing an immune response modulator in an animal.

### PRIORITY

Priority is claimed to U.S. Provisional Application Ser. No. U.S. 60/857,663, filed Nov. 7, 2006, and entitled Virus-mediated Cytoplasmic Expression of DNA Plasmid Vaccines,” which is referred to and incorporated herein by reference in its entirety.

### FIELD OF THE INVENTION

The invention pertains generally to novel methods and compositions for cytoplasmic delivery and expression of DNA vaccines. The methodology involves co-administration of a replication-competent or defective vesicular stomatitis virus (VSV) vector expressing a bacteriophage RNA polymerase enzyme and one or more DNA plasmids encoding any variety of antigens under control of the cognate bacteriophage promoter DNA sequence. The present invention further relates to methods, systems, and compositions for expressing an immune response modulator in an animal

### BACKGROUND

DNA cloning technology provides a readily amplifiable source of genes encoding any protein of interest. When the recombinant protein itself is needed, genes are cloned in expression vectors that are introduced into appropriate cell types where protein synthesis can take place. To produce large amounts of these proteins, two general types of transient gene expression vectors have been used: plasmid DNA vectors which are introduced directly into cells and viral vectors that express foreign genes as part of their genetic material. The latter type of vector is generally more efficient in higher eukaryotic cells because all cells can be infected simultaneously and many viruses can express proteins at very high levels. Plasmid vector preparation is less labor intensive, but DNA transfection can be inherently less efficient and amounts of protein synthesized are generally lower.

High-level recombinant protein expression is crucial for the biopharmaceutical industry as well as for basic research. Large amounts of specific proteins are very often required for general biochemical characterization, structural studies, drug discovery development, gene therapy, subunit vaccine production, and reagent use. Different uses dictate which particular protein expression system provides the best combination of properties. For example, high-level transient protein expression in mammalian cells most often makes use of viral vectors (e.g., adenovirus, baculovirus, poxvirus, alphavirus). In most applications, the gene of interest is cloned into the virus genome or a derivative replicon which is labor intensive and time consuming. Plasmid vectors are also used for transient protein expression but efficiency is generally much lower. Another method employs recombinant viruses that express the T′7 RNA polymerase to drive expression of desired proteins from plasmids under control of a T7 promoter. This latter approach is very efficient using a vaccinia-T7 recombinant virus especially when incorporating an internal ribosome entry sequence (IRES) in the T7 transcript. However, high level protein production using the vaccinia-T7 system is limited to host cells that grow the virus efficiently. Moreover, the use of an infectious virus related to the smallpox vaccine strain raises biosafety concerns.

Vesicular stomatitis virus (VSV), of the genus, Vesiculovirus, is the prototypic member of the family Rhabdoviridae, and is an enveloped virus with a negative stranded RNA genome that causes a self-limiting disease in live-stock and is essentially non-pathogenic in humans. (Balachandran and Barber, 2000, IUBMB Life 50: 135-8). Rhabdoviruses have single, negative-strand RNA genomes of 11,000 to 12,000 nucleotides (Rose and Schubert, 1987, Rhabdovirus genomes and their products, in The Viruses: The Rhabdoviruses, Plenum Publishing Corp., NY, pp. 129-166). The virus particles contain a helical, nucleocapsid core composed of the genomic RNA and protein. Generally, three proteins, termed N (nucleocapsid, which encases the genome tightly), P (formerly termed NS, originally indicating nonstructural), and L (large) are found to be associated with the nucleocapsid. An additional matrix (M) protein lies within the membrane envelope, interacting both with the membrane and the nucleocapsid core. A single glycoprotein (G) species spans the membrane and forms the spikes on the surface of the virus particle. Glycoprotein G is responsible for binding to cells and membrane fusion. The VSV genome is the negative sense (i.e., complementary to the RNA sequence (positive sense) that functions as mRNA to directly produce encoded protein), and rhabdoviruses must encode and package an RNA-dependent RNA polymerase in the virion (Baltimore et al., 1970, Proc. Natl. Acad. Sci. USA 66: 572-576), composed of the P and L proteins. This enzyme transcribes genomic RNA to make subgenomic mRNAs encoding the 5-6 viral proteins and also replicates full-length positive and negative sense RNAs. The genes are transcribed sequentially, starting at the 3′ end of the genomes.

VSV replicates rapidly (<12 hours) and very efficiently in the cytoplasm of almost all vertebrate cells and produces very high levels of infectious virus (titers approaching 20,000 infectious units/cell in some cases) and can also infect insect cells. The sequences of the VSV mRNAs and genome are described in, for example, Gallione et al. 1981, R Virol. 39: 529-535; Rose and Gallione, 1981, J. Virol. 39: 519-528; Rose and Schubert, 1987; Rhabdovirus genomes and their products, p. 129-166, in R. R. Wagner (ed.); The Rhabdoviruses, Plenum Publishing Corp., NY; Schubert et al., 1985, Proc. Natl. Acad. Sci. USA 82: 7984-7988. WO96/34625 published Nov. 7, 1996, discloses methods for the production and recovery of replicable vesiculovirus. U.S. Pat. No. 6,168,943, issued Jan. 2, 2001, describe methods for making recombinant vesiculoviruses.

Growth in tissue culture and purification of virus is relatively simple, and methods for engineering mutations or additional genes in the virus genome, while retaining very high infectivity, are well established. It is, moreover, possible to engineer the surface protein of the virus and target infection to specific cell types. Natural hosts include cattle, horses, and pigs where it causes a non-fatal but debilitating disease. Laboratory strains pose very little if any risk of pathogenicity in humans. VSV is currently being explored as a vector for vaccine production, gene replacement therapy, and anticancer therapy.

Expression of the T7 RNA polymerase enzyme by recombinant viruses has been reported (see e.g. Mohammed et al., Methods Mol. Biol. 2004; 269:41-50; and Eckert et al., J Gen Virol. 1999 June; 80 (Pt 6):1463-9). Recombinant T7-expressing viruses are capable of driving transient expression of proteins from plasmids. The best characterized of these systems is the recombinant vaccinia-T7 virus which yields very high levels of protein. VSV expression of T7 RNA polymerase enzyme as part of an expression system is discussed in WO2005/117557 by Jacques Perrault, published Dec. 15, 2005, which is hereby incorporated by reference herein in its entirety. There is a need for a safe and efficient virus-bacteriophage RNA polymerase vaccination system.

### DESCRIPTION

In one embodiment of the invention is provided a system for expressing immune response modulators. Also provided is a system for expressing one, two, or more heterologous sequences in a cell. The expression systems provided comprise combination of a virus vector and a plasmid that leads to very high transient expression of heterologous polynucleotides and polypeptides. The systems may be used for example, for DNA vaccination. Methods for making and using the systems are also provided.

Vesicular stomatitis virus is engineered to express bacteriophage RNA polymerase enzyme in the cytoplasm of infected cells. Infected cells are then transfected with a plasmid DNA encoding a gene of interest downstream of a corresponding bacteriophage promoter sequence, which may comprise an internal ribosome entry sequence. By “corresponding” is meant that the bacteriophage RNA polymerase expressed by the VSV vector acts on the bacteriophage promoter sequence, so that, for example, where the bacteriophage RNA polymerase is SP6 enzyme, the corresponding promoter sequence is a SP6 promoter. This results in cytoplasmic accumulation of large amounts of mRNA transcripts which are efficiently translated into the desired protein. Methods and compositions are also provided bypassing the need for an internal ribosome entry sequence in the transfected plasmid.

It is to be understood by those of ordinary skill in the art that the methods and systems herein are not limited to VSV, but may also be applied to other rhabdoviruses. Thus any Rhabdovirus (human, animal, plant) may be engineered to express a bacteriophage RNA polymerase, thus the viruses may be used as vectors in the appropriate host. Thus, using the appropriate Rhabdovirus, fish vaccines may be used, useful proteins may be produced in plants, transgenic mosquitos may be created, and the like.

Provided herein are compositions and methods for a recombinant vesicular stomatitis virus vector expression system. Provided also are compositions and methods for DNA vaccines comprising a vesicular stomatitis virus vector expression system. A vesicular stomatitis virus vector particle (VSV) encoding a bacteriophage RNA polymerase is provided and used to infect a cell, such that the polymerase is expressed in the cell. Also provided is a recombinant plasmid vector encoding a heterologous gene under control of a corresponding bacteriophage promoter and also may encode an IRES element. Cells infected with VS V-bacteriophage RNA polymerase viral particles are subsequently, or concurrently transfected with the recombinant plasmid vector such that a polynucleotide is expressed from the transcripts encoded by the heterologous gene in the cell. A method for producing a polypeptide by contacting cells with the recombinant vesicular stomatitis virus vector expression system is also provided. In one embodiment, the VSV-bacteriophage RNA polymerase viral particles include the sequences of polynucleotides as set forth in SEQ ID NO. 1. In another embodiment, the M and G genes of the VSV-bacteriophage RNA polymerase vector virus vector particle are deleted or mutated such that the virus vector particle is replication-deficient. A recombinant plasmid vector consisting essentially of the polynucleotide sequence as set forth in SEQ ID NO. 3 are also provided.

In one aspect, is provided, a vesicular stomatitis virus (VSV) expression system, comprising a recombinant plasmid vector comprising a polynucleotide comprising the following elements operably linked 5′ to 3′: (i) a bacteriophage promoter sequence, and (ii) at heterologous gene; and a vesicular stomatitis virus vector particle (VSV) comprising a polynucleotide encoding a bacteriophage RNA polymerase that operates on said bacteriophage promoter sequence. The vesicular stomatitis virus vector particle may, for example, comprise the polynucleotide sequence of SEQ ID No. 1.

The bacteriophage RNA polymerase may be, for example, be any RNA polymerase belonging to the T7-like group or T7 supergroup. For example, the RNA polymerase may be selected from the group consisting of T7, SP6, T1, T3, and T5 RNA polymerases and said bacteriophage RNA polymerase promoter may be, for example, selected from the group consisting of T7, SP6, T1, T3 and T5 promoters. Where the promoter is a T7 promoter, the T7 promoter may, for example, comprise a T7 promoter corresponding to residues 794 to 813 of SEQ ID No. 3. Those of ordinary skill in the art understand that such plasmid vectors may be circular or linear, and may, for example, comprise chemically modified DNA. The RNA polymerase may also be modified, and these modified RNA polymerases are within the scope of the invention.

T7 transcripts lack the methylated caps required for translation in eukaryotes (T7 polymerase is prokaryotic) Vaccinia virus encodes capping and cap methylation enzymes that function in trans to modify T7 transcripts. VSV capping and methylation enzymes are cis-acting. The cap requirement can be bypassed by using an internal ribosome entry element (IRES) as in the pTM1 plasmid. Any trans-acting capping enzymes known to those of ordinary skill in the art, encoded by other viruses or cells, may also be used to cap the transcripts.

The heterologous gene may, for example, comprise a sequence encoding an internal ribosome entry site (IRES). Also, the system may further comprise a DNA sequence encoding a vaccinia capping enzyme. For example, the system may comprise a first DNA sequence encoding the D1 catalytic subunit of vaccinia capping enzyme, and a second DNA sequence encoding the D12 subunit of vaccinia capping enzyme. These DNA sequences may, for example, be present on one plasmid vector, called herein a recombinant plasmid capping vector, or the DNA sequences may be present on two different recombinant plasmid capping vectors. The D1 catalytic subunit sequence may comprise, for example, the sequence set forth in SEQ ID NO. 5, and the D12 catalytic subunit sequence may comprise, for example, the sequence set forth in SEQ ID NO. 6. The system may also be used to deliver interfering RNAs.

In other system embodiments, the bacteriophage promoter sequence may be operably linked to a bacteriophage gene expression regulator sequence. This regulator sequence may include, for example, any DNA sequence that enhances, increases, decreases, or otherwise modulates the expression of a gene linked to the bacteriophage promoter sequence. The presence of the DNA sequence itself may modulate this expression, or, for example, a transcript, or a protein encoded by the DNA sequence may modulate the expression. In some examples, the regulator sequence may be, for example, selected from the group consisting of a ribo-switch sequence, and a ligand-regulated protein binding site. Ligand-regulated protein binding sites include, for example, inducible systems such as the classic lac repressor-operator example, and many other well characterized inducible systems.

In yet other embodiments, the system further comprises a DNA sequence encoding a protein that regulates bacteriophage RNA polymerase activity. For example, the system may comprise a DNA sequence encoding a lysozyme.

In other embodiments, the system may comprise a DNA sequence encoding a protein that modulates the immune response of a host animal. Any immune response modulator known to those of ordinary skill in the art may be used. The host animal may be, for example a mammal, for example, a human. Proteins that may modulate the immune response of a host animal include, for example, a protein selected from the group consisting of an enhancer of antigen presentation to APCs (e.g. GM-CSF), a factor that helps recruit and activate dendritic cells (DCs) (e.g. MIP-1α or Flt3L), an enhancer of T-lymphocyte priming (e.g. B7-2 or CD154), and a stimulator of T-lymphocyte expansion (e.g. IL-2, IL-12, or IL-15).

In certain embodiments, the system may provide components for producing two or more heterologous proteins in a cell. For example, the recombinant plasmid vector may comprise a DNA sequence encoding two or more heterologous proteins operably linked to the bacteriophage promoter sequence. For example, said recombinant plasmid vector may comprise a DNA sequence encoding two, three, four, five, six, or seven heterologous proteins. In some examples, the DNA sequence encoding two or more heterologous proteins comprises two or more sequences encoding internal ribosome entry sites, each of which enables translation of a different heterologous protein. In other examples, the DNA sequence comprises a heterologous gene that encodes a fusion protein comprising two or more heterologous proteins. In some examples, the plasmid comprises two or more bacteriophage promoter sequences, and said system further comprises a DNA sequence encoding a DNA endonuclease, capable of expression in a cell and releasing individual expression cassettes from said plasmid, said expression cassettes comprising a bacteriophage promoter sequence and a DNA sequence encoding a heterologous protein. In other examples, the system further comprises a DNA sequence encoding a ribozyme, said ribozyme is capable of expression in a cell, and cleaving transcripts encoding said two or more heterologous proteins to provide two or more individual transcripts, each of which codes for a different heterologous protein.

Also provided in the present invention are systems for producing two or more heterologous proteins in a cell, comprising two or more recombinant plasmid vectors, each comprising a polynucleotide comprising the following elements operably linked 5′ to 3′: (i) a bacteriophage promoter sequence, and (ii) a DNA sequence encoding a heterologous protein; and a vesicular stomatitis virus vector particle (VSV) comprising a polynucleotide encoding a bacteriophage RNA polymerase that operates on said bacteriophage promoter sequence.

In some examples, systems are provided comprising two, three, four, five, six, seven, eight, nine, or ten recombinant plasmid vectors, each comprising a polynucleotide comprising the following elements operably linked 5′ to 3′: (i) a bacteriophage promoter sequence, and (ii) a DNA sequence encoding a heterologous protein.

The present invention provides DNA vaccines. Thus, also provided in the present invention are vesicular stomatitis expression systems formulated for injection into a host. These systems are therefore formulated for administration to an animal, for example a mammal, for example, a human. Various routes of administration are provided in the present invention, including, for example, intra-muscular injection, intra-peritoneal injection, intravenous delivery, subcutaneous deliver, intra-nasal delivery, and oral delivery. The systems thus may further comprise an agent that promotes entry of DNA into cells. This agent may, for example, be selected from the group consisting of lipids, polymers, and gold particles.

Also provided are systems of the present invention formulated for transfecting cells by a method selected from the group consisting of liposome mediated transfer, lipofection, polycation-mediated transfer, direct DNA transfer, electroporation, gene gun use, transfection in the presence of polymers, and transfection in the presence of gold particles. Transfection methods include those, for example, involving lipids (e.g., cationic lipids, liposomes), cationic polymers (e.g., polyethylene imine, DEAE dextran), cationic salts (e.g., calcium phosphate), peptides or small proteins, peptides or small proteins mixed with lipids, nanoparticles of many different compositions (e.g., gold), dendrimers, electroporation, laser-assisted electroporation, gene gun, and direct injection.

The VSV vector of the present systems may, for example, encode a heterologous protein or a modified vesicular stomatitis virus surface protein that reduces immunity to vesicular stomatitis virus vector on subsequent deliveries of a vesicular stomatitis virus vector. The VSV vector may, for example, encode heterologous protein or a modified vesicular stomatitis virus surface protein that causes targeting of said virus vector to receptors on a specific cell type.

Also provided are VSV expression systems of the present invention wherein the VSV vector is propagation defective. These replication-deficient VSV vectors are known to those of ordinary skill in the art, and include, for example, but are not limited to, VSV M-deficient and VSV M plus G-deficient vectors. For example, the M and G genes of the virus vector may be deleted or mutated such that the virus vector particle is replication-deficient. To produce such virus vectors, the virus vector particle may be produced by, for example, transfecting a permissive producer cell with a vector comprising a nucleic acid sequence of at least part of the VSV genome and T7 RNA polymerase wherein the M and G genes are deleted, growing said the producer cell under cell culture conditions sufficient to allow producing of vesicular stomatitis virus vector particles in said cell, co-transfecting said cell with plasmids encoding M and G genes; and collecting said particles. In some examples, the producer cell is grown in cell culture medium, and the replication-defective vector particles are collected from the medium. In other examples, the replication-defective vector particles are collected from the producer cells. The VSV M protein rapidly blocks host transcription and mRNA transport to the cytoplasm. M protein also inhibits host cell mRNA translation (mechanism unknown). Wild-type M protein may thus compromise translation of T7 transcripts. One solution, for example, is to engineer M protein mutations that reduce host cell shutoff.

Also provided are methods for using the systems of the present invention to express heterologous proteins in cells, for example, by contacting said cells with the viruses and vectors of the present systems. Also provided are methods for vaccinating an animal, for example a human, using the present systems. In other embodiments are provided vaccines comprising the systems of the present invention. In these embodiments, the components of a system of the present invention may be provided in, for example, one or multiple vaccines. That is, for example, where vaccination is by injection, one injection may be provided comprising all of the components of a system of the present invention. Or, for example, the components may be distributed into more than one injection, for example, the vesicular stomatitis virus vector may be provided in one injection, and a recombinant plasmid may be provided in another injection.

The invention also provides any of the above methods for expressing proteins or delivering interfering RNA wherein the cells are transfected with said recombinant plasmid vectors by a suitable method such as liposome mediated transfer, lipofection, polycation-mediated transfer, or direct DNA transfer or uptake. The expression of a gene product in a cell may be reduced as a result of expression of an interfering RNA polynucleotide.

The details of one or more embodiments of the invention are set forth in the accompanying drawings and the description below. Other features, objects, and advantages of the invention will be apparent from the description and drawings, and from the claims. These and other embodiments are described hereafter.

### BRIEF DESCRIPTION OF THE DRAWINGS

FIG. 1 depicts a pVSV-T7 plasmid map.

FIG. 2 depicts a pVSV-GFP plasmid map.

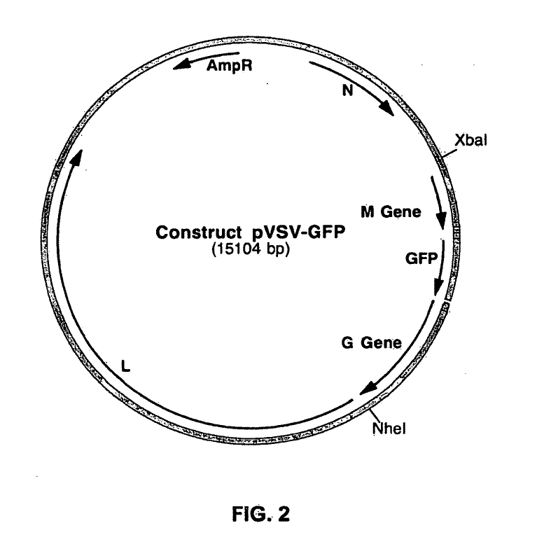

FIG. 3 depicts a pTM1-GFP plasmid map.

FIG. 4 depicts a pSP73-GFP plasmid map.

FIG. 5 depicts a pTM1-D1 plasmid map.

FIG. 6 depicts a pTM1-D12 plasmid map.

FIG. 7 represents a schematic of a vesicular stomatitis virus; structural genes are shown as is a schematic of the VSV genome.

FIG. 8 represents a schematic of the VSV genome, including the sequences of the leader-N gene junction and other gene junctions. Unique features of VSV transcription are also provided.

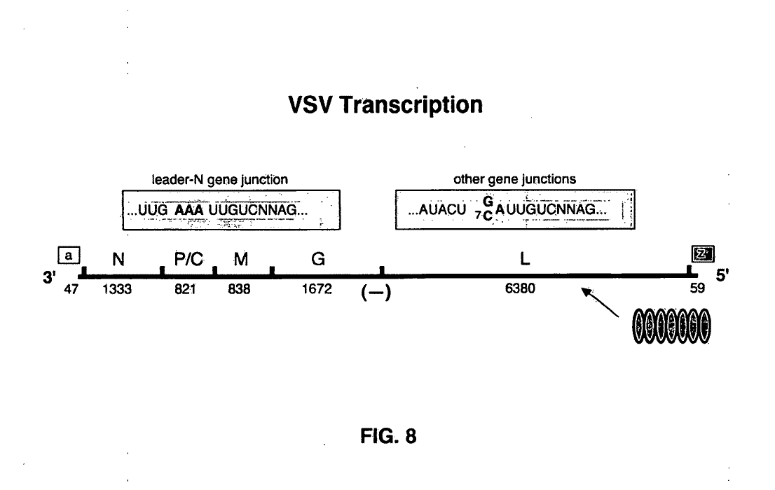

FIG. 9 represents a schematic of general features of VSV RNA synthesis.

FIG. 10 represents a schematic VSV RNA synthesis emphasizing the role of the P, N and L VSV gene products during transcription.

FIG. 11 represents a schematic of an engineered VSV genome; any foreign gene flanked by gene end and start signals are faithfully transcribed by the viral polymerase.

FIG. 12 represents a schematic of the VSV-T7 RNA polymerase construct as described herein.

FIG. 13 shows expression of GFP in cells transfected with the vaccinia T7 system and pTM1-GFP plasmid encoding an IRES element.

FIG. 14 shows expression of GFP in cells transfected with the vaccinia T7 system and pSP73-GFP plasmid without an IRES element.

FIG. 15 shows expression of GFP in cells transfected with the VSV-T7 system and pTM1-GFP plasmid encoding an IRES element.

FIG. 16 shows expression of GFP in cells transfected with the VSV-T7 system and either the pSP73-GFP (no IRES) or pTM1-GFP (with IRES) plasmids.

FIG. 17 shows comparison of expression of GFP in cells transformed with either the VSV-T7 or vaccinia-T7 systems.

FIG. 18 shows expression of GFP in cells with the VSV-T7 system and co-transfected with plasmids pTM1-D1 and pTM1-D12 that encode the vaccinia capping enzyme.

FIG. 19 shows a comparison of expression of the firefly luciferase gene in place of GFP for the VSV-T7 versus vaccinia-T7 system.

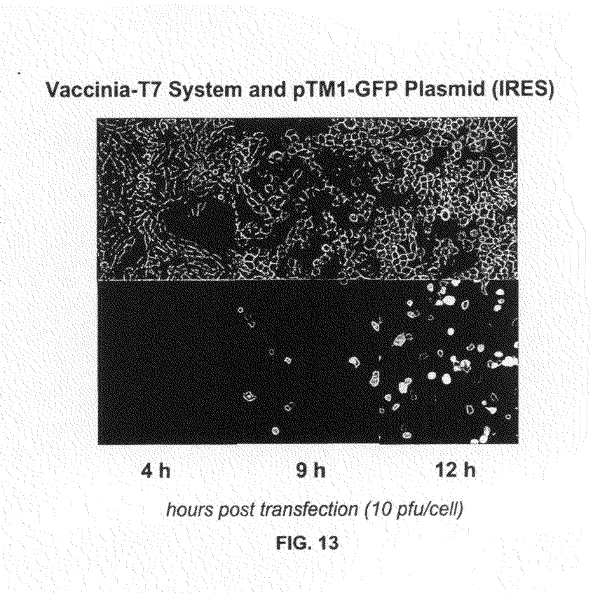

For FIGS. 13-19, the experiments made use of BHK cell monolayers in 5 cm dishes. A homemade “transfectace” (cationic lipid transfection reagent) was used in all cases. The virus multiplicity of infection (m.o.i.) was 10 pfu/cell unless otherwise stated.

### DETAILED DESCRIPTION

### A. Definitions

Unless defined otherwise, all technical and scientific terms used herein have the same meaning as is commonly understood by one of skill in the art to which the subject matter described herein belongs.

As used herein, the terms “heterologous” or “foreign nucleic acid” are used interchangeably and refer to DNA or RNA that does not occur naturally as part of the genome in which it is present or which is found in a location or locations in the genome that differs from that in which it occurs in nature. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is expressed. These are referred to herein as heterologous RNAs and heterologous proteins. Any DNA or RNA that one of skill in the art would recognize or consider as heterologous or foreign to the cell in which it is expressed is herein encompassed by the term heterologous DNA. Examples of heterologous DNA include, but are not limited to, DNA that encodes transcriptional and translational regulatory sequences and selectable or traceable marker proteins, such as a protein that confers drug resistance. Heterologous DNA may also encode DNA that mediates or encodes mediators that alter expression of endogenous DNA by affecting transcription, translation, or other regulatable biochemical processes.

As used herein, the term “vector” refers to a polynucleotide construct designed for transduction/transfection of one or more cell types. Selection and use of such vectors are well within the level of skill of the art. Generally, vectors are derived from viruses or plasmids of bacteria and yeasts. As used herein, VSV vectors may be, for example, “cloning vectors” which are designed for isolation, propagation and replication of inserted nucleotides, “expression vectors” which are designed for expression of a nucleotide sequence in a host cell, a “viral vector” which is designed to result in the production of a recombinant virus or virus-like particle, or “shuttle vectors”, which comprise the attributes of more than one type of vector. The present invention encompasses VSV vectors that comprise nucleic acid encoding viral structural proteins capable of assembling into virus-like particles. As used herein, in the context of VSV, a “heterologous polynucleotide” or “heterologous gene” or “transgene” is any polynucleotide or gene that is not present in wild-type VSV. As used herein, in the context of VSV, a “heterologous” promoter is one which is not associated with or derived from VSV.

As used herein, “expression” refers to the process by which nucleic acid is transcribed into mRNA and translated into peptides, polypeptides, or proteins. “Expression” may be characterized as follows: a cell is capable of synthesizing many proteins. At any given time, many proteins which the cell is capable of synthesizing are not being synthesized. When a particular polypeptide, coded for by a given gene, is being synthesized by the cell, that gene is said to be expressed. In order to be expressed, the DNA sequence coding for that particular polypeptide must be properly located with respect to the control region of the gene. The function of the control region is to permit the expression of the gene under its control. As used herein, the term “expression vector” includes vectors capable of expressing DNA or RNA fragments that are in operative linkage with regulatory sequences, such as promoter regions, that are capable of effecting expression of such DNA or RNA fragments. Thus, an expression vector refers to a recombinant DNA or RNA construct, such as a plasmid, a phage, recombinant virus or other vector that, upon introduction into an appropriate host cell, results in expression of the cloned DNA or RNA. Appropriate expression vectors are well known to those of skill in the art and include those that are replicable in eukaryotic cells and/or prokaryotic cells and those that remain episomal or may integrate into the host cell genome.

As used herein, the terms “operative linkage” or “operative association” of heterologous DNA to regulatory and effector sequences of nucleotides, such as promoters, enhancers, transcriptional and translational stop sites, and other signal sequences, refer to the functional relationship between such DNA and such sequences of nucleotides. For example, operative linkage of heterologous DNA to a promoter refers to the physical and functional relationship between the DNA and the promoter such that the transcription of such DNA is initiated from the promoter by an RNA polymerase that specifically recognizes, binds to and correctly transcribes the DNA.

As used herein, the term “promoter region” refers to the portion of DNA of a gene that controls transcription of DNA to which it is operatively linked. A portion of the promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated. For use herein, inducible promoters are preferred. The promoters are recognized by an RNA polymerase that is expressed by the host. The promoter may be any bacteriophage promoter that can be recognized by an RNA polymerase that is expressed in the host. The promoter may be, for example, recognized by T7, SP6, T1, T3, or T5 RNA polymerases.

As used herein, “RNA polymerase” may be endogenous to the host or may be introduced by genetic engineering into the host, either as part of the host chromosome or on an episomal element, including a plasmid containing the DNA encoding an RNA polymerase. The RNA polymerase may be, for example, T7, SP6, T1, T3, or T5 RNA polymerase.

As used herein, the term “transcription terminator region” has (a) a subsegment that encodes a polyadenylation signal and polyadenylation site in the transcript, and/or (b) a subsegment that provides a transcription termination signal that terminates transcription by the polymerase that recognizes the selected promoter. The entire transcription terminator may be obtained from a protein-encoding gene, which may be the same or different from the gene, which is the source of the promoter. Transcription terminator regions can be those that are functional in E. coli. Transcription terminators are optional components of the expression systems herein, but are employed in preferred embodiments.

As used, the term “nucleotide sequence coding for expression of” or “encoding” a polypeptide refers to a sequence that, upon transcription and subsequent translation of the resultant mRNA, produces the polypeptide.

As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, maintenance of the correct reading frame of a protein-encoding gene to permit proper translation of the mRNA, and stop codons. In addition, sequences of nucleotides encoding a fluorescent indicator polypeptide, such as, for example, a green or blue fluorescent protein, or, for example, a luciferase protein, can be included in order to select positive clones (i.e., those host cells expressing the desired polypeptide).

As used herein, “host cells” or “target cells” are cells in which a vector can be propagated and its nucleic acid expressed. The term also includes any progeny of the subject host cell. It is understood that all progeny may not be identical to the parental cell since there may be mutations that occur during replication. Such progeny are included when the term “host cell” is used. A “host cell” includes an individual cell or cell culture which can be or has been a recipient of a VSV vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected, (no permanent genetic change is possible in this system) or infected in vivo or in vitro with a VSV vector of this invention.

As used herein, the term “packaging cell” or “packaging cell line” refers to a cell or cell lines that are able to package viral genomes or modified genomes or its equivalents. Thus, packaging cells can provide complementing functions for any genes deleted in a viral genome (e.g. nucleic acids encoding structural genes) and are able to package the viral genomes into viral vector particles. The production of such particles requires that the genome be replicated and that those proteins necessary for assembling a viral particle (infectious or non-infectious) are produced. The particles also can require certain proteins necessary for the maturation of the viral particle. Such proteins can be provided by the vector or by the packaging cell.

As used herein, the term “transfection” refers to the taking up of DNA or RNA by a host cell. Transformation refers to this process performed in a manner such that the DNA is replicable, either as an extrachromosomal element or as part of the chromosomal DNA of the host. Methods and means for effecting transfection and transformation are well known to those of skill in this art (see, e.g., Wigler et al. (1979) Proc. Natl. Acad. Sci. USA 76:1373-1376; Cohen et al. (1972) Proc. Natl. Acad. Sci. USA 69:2110). Cells may be transfected in vitro, or may be transfected after administration of the DNA or RNA to a host, for example, by injection.

As used herein, the term “transformation” refers to the transfer of a nucleic acid fragment into the genome of a host cell, resulting in genetically stable inheritance. A “host cell” is a cell that has been transformed, or is capable of transformation, by an exogenous nucleic acid molecule. Host cells containing the transformed nucleic acid fragments are referred to as “transgenic” cells, and organisms comprising transgenic cells are referred to as “transgenic organisms”.

As used herein, the term “isolated substantially” pure DNA refers to DNA fragments purified according to standard techniques employed by those skilled in the art (see, e.g., Maniatis et al. (1982) Molecular Cloning: A Laboratory Manual, Cold Spring Harbor Laboratory Press, Cold Spring Harbor, N.Y. and Sambrook et al. (1989) Molecular Cloning: A Laboratory Manual, Cold Spring Harbor Laboratory Press, Cold Spring Harbor, N.Y.).

As used herein, a “culture” means a propagation of cells in a medium conducive to their growth, and all sub-cultures thereof. The term subculture refers to a culture of cells grown from cells of another culture (source culture), or any subculture of the source culture, regardless of the number of subculturings that have been performed between the subculture of interest and the source culture.

The term “to culture” refers to the process by which such culture propagates. As used herein, the term “peptide” and/or “polypeptide” means a polymer in which the monomers are amino acid residues which are joined together through amide bonds, alternatively referred to as a polypeptide. When the amino acids are alpha-amino acids, either the L-optical isomer or the D-optical isomer can be used, the L-isomers being preferred. Additionally, unnatural amino acids such as beta-alanine, phenylglycine, and homoarginine are meant to be included. Standard single and three letter naming conventions for amino acids are used herein.

As used herein, the term “restriction enzyme digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 microgram of plasmid or DNA fragment is used with about 1-2 units of enzyme in about 20 microliters of buffer solution. Appropriate buffers and substrate amounts for particular restriction enzymes are specified by the manufacturer. Incubation of about 1 hour at 37° C. is ordinarily used, but may vary in accordance with the supplier's instructions. After incubation, protein or polypeptide is removed by extraction with phenol and chloroform, and the digested nucleic acid is recovered from the aqueous fraction by precipitation with ethanol. Digestion with a restriction enzyme may be followed with bacterial alkaline phosphatase hydrolysis of the terminal 5′ phosphates to prevent the two restriction cleaved ends of a DNA fragment from circularizing or forming a closed loop that would impede insertion of another DNA fragment at the restriction site. Unless otherwise stated, digestion of plasmids is not followed by 5′ terminal dephosphorylation. Procedures and reagents for dephosphorylation are conventional as described in Sections 1.56-1.61 of Sambrook, et. al., Molecular Cloning: A Laboratory Manual New York: Cold Spring Harbor Laboratory Press, 1989 (which disclosure is hereby incorporated by reference).

The terms “recovery” or “isolation” of a given fragment of DNA from a restriction digest mean separation of the digest, e.g., on polyacrylamide or agarose gel by electrophoresis, identification of the fragment of interest by comparison of its mobility versus that of marker DNA fragments of known molecular weight, removal of the gel section containing the desired fragment, and separation of the gel from DNA. These procedures are generally well known. For example, see Lawn et al., 1981, Nucleic Acids Res., vol. 9, pp. 6103-6114; and Goeddel et al., 1980, Nucleic Acids Res., vol. 8, p. 4057, which disclosures are hereby incorporated by reference.

As used herein, the term “gene” refers to those DNA sequences which transmit the information for and direct the synthesis of a single protein chain.

As used herein, “gene therapy” refers to genetic therapy that involves the transfer of heterologous DNA to certain cells, target cells, of a mammal, particularly a human, with a disorder or condition for which such a therapy is sought. The DNA is introduced into the selected target cells in a manner such that the heterologous DNA is expressed and a therapeutic product encoded thereby is produced. Alternatively, the heterologous DNA can in some manner mediate expression of DNA that encodes the therapeutic product, it can encode a product, such as a peptide or RNA that in some manner mediates, directly or indirectly, expression of a therapeutic product. Gene therapy also can be used to deliver nucleic acid encoding a gene product to replace a defective gene or supplement a gene product produced by the mammal or a cell in which it is introduced. The introduced nucleic acid can encode a therapeutic compound, such as a growth factor or inhibitor thereof, or a tumor necrosis factor or inhibitor thereof, or a receptor thereof, that is not normally produced in the mammalian host or that is not normally produced in therapeutically effective amounts or at a therapeutically useful time. The introduced nucleic acid can encode, for example, an immune response modulator. The heterologous DNA encoding the therapeutic product can be modified prior to introduction into the cells of the afflicted host to enhance or otherwise alter the product or expression thereof.

As used herein, “therapeutic nucleic acid” refers to a nucleic acid that encodes a therapeutic product. The product can be nucleic acid, such as a regulatory sequence or gene, or can be a protein that has a therapeutic activity or effect. For example, a therapeutic nucleic acid can be a ribozyme, antisense, double-stranded RNA, a nucleic acid encoding a protein or otherwise.

As used herein, an immune response modulator refers to any factor, such as, for example, a protein, peptide, or nucleic acid, that modulates, regulates, or otherwise changes the immune system of an animal, for example, a human. A non-limiting list of examples include modulators known to those of ordinary skill in the art, including, for example, but not limited to, APCs (e.g. GM-CSF), a factor that helps recruit and activate DCs (e.g. MIP-1α or Flt3L), an enhancer of T-lymphocyte priming (e.g. B7-2 or CD154), and a stimulator of T-lymphocyte expansion (e.g. IL-2, IL-12, or IL-15).

As used herein, the term “infection” refers to the invasion by agents (e.g., viruses, viral vector particles, bacteria, etc.) of cells where conditions are favorable for their replication and growth.

As used herein, the term “plasmid” means a vector used to facilitate the transfer of exogenous genetic information, such as the combination of a promoter and a heterologous gene under the regulatory control of that promoter. The plasmid can itself express a heterologous gene inserted therein. “Plasmids” are designated by a lower case p preceded and/or followed by capital letters and/or numbers. The starting plasmids herein are commercially available, are publicly available on an unrestricted basis, or can be constructed form such available plasmids in accord with published procedures. In addition, other equivalent plasmids are known in the art and will be apparent to one of ordinary skill in the art.

The term “ligation” means the process of forming phosphodiester bonds between two nucleic acid fragments. To ligate the DNA fragments together, the ends of the DNA fragments must be compatible with each other. In some cases, the ends will be directly compatible after endonuclease digestion to blunt ends to make them compatible for ligation. To blunt the ends, the DNA is treated in a suitable buffer for at least 15 minutes at 15° C., with about 10 units of the Klenow fragment of DNA polymerase I or T4 DNA polymerase in the presence of the four deoxyribonucleotide triphosphates. The DNA is then purified by phenolchloroform extraction and ethanol precipitation. The DNA fragments that are to be ligated together are put in solution in about equimolar amounts. The solution will also contain ATP, ligase buffer, and a ligase such as T4 DNA ligase at about 10 units per 0.5 microgram of DNA. If the DNA is to be ligated into a vector, the vector is first linearized by digestion with the appropriate restriction endonuclease(s). The linearized fragment is then treated with bacterial alkaline phosphatase, or calf intestinal phosphatase to prevent self-ligation during the ligation step.

As used herein, the term “preparation of DNA from cells” means isolating the plasmid DNA from a culture of the host cells. Commonly used methods for DNA preparation are the large and small scale plasmid preparations described in sections 1.25-1.33 of Sambrook et al., supra, which disclosure is hereby incorporated by reference. After preparation of the DNA, it can be purified by methods well known in the art such as that described in section 1.40 of Sambrook et al., supra, which disclosure is hereby incorporated by reference.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to one or more of SEQ ID NOs. 1-6) also includes the complementary sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

As used herein, the term “under transcriptional control” refers to a term well understood in the art and indicates that transcription of a polynucleotide sequence depends on its being operably (operatively) linked to an element which contributes to the initiation of, or promotes, transcription. “Operably linked” refers to a juxtaposition wherein the elements are in an arrangement allowing them to function.

“Replication” and “propagation” are used interchangeably and refer to the ability of an VSV vector of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

As used herein, “expression” includes transcription and/or translation of a polynucleotide sequence.

As used herein, “internal ribosome entry sequence” (“IRES”) refers to nucleic acid sequences which exhibit IRES activity (IRES elements), i.e. sequences which are capable of providing cap-independent translation of a downstream gene or coding sequence by an internal ribosome entry mechanism.

As used herein, “functional RNA” refers to sense RNA, antisense RNA, ribozyme RNA, siRNA, or other RNA that may not be translated but yet has an effect on at least one cellular process.

As used herein, “altered levels” refers to the level of expression in transgenic cells or organisms that differs from that of normal or untransformed cells or organisms.

As used herein, a first sequence is an “antisense sequence” with respect to a second sequence if a polynucleotide whose sequence is the first sequence specifically binds to, or specifically hybridizes with, the second polynucleotide sequence under physiological conditions.

As used herein, “antisense inhibition” refers to the production of antisense RNA transcripts capable of suppressing the expression of protein from an endogenous gene or a transgene.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent conditions” or “high stringency conditions”, as defined herein, are identified by, but not limited to, those that (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 micrograms/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. “Moderately stringent conditions” are described by, but not limited to, those in Sambrook et al., supra, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

Polynucleotides are described as “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree that one polynucleotide is complementary with another) is quantifiable in terms of the proportion of bases in opposing strands that are expected to form hydrogen bonds with each other, according to generally accepted base-pairing rules.

As used herein, “RNA interference (RNAi)” is the process of sequence-specific, post-transcriptional gene silencing initiated by double stranded RNA (dsRNA) or siRNA. RNAi is seen in a number of organisms such as Drosophila, nematodes, fungi and plants, and is believed to be involved in anti-viral defense, modulation of transposon activity, and regulation of gene expression. During RNAi, dsRNA or siRNA induces degradation of target mRNA with consequent sequence-specific inhibition of gene expression.

As used herein, “interfering RNA” and “small interfering RNA” (siRNA) refer to a RNA duplex of nucleotides that is targeted to a gene of interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ and/or 5′ overhang portions. In some embodiments, the overhang is a 3′ and/or a 5′ overhang 1, 2, 3, 4 or 5 nucleotides in length.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

The practice of the present invention will employ, unless otherwise indicated, conventional techniques of molecular biology (including recombinant techniques), microbiology, cell biology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

### B. VSV-T7 RNA Polymerase Polypeptide Expression System

1. VSV

For general, information related to vesicular stomatitis virus, see, “Fundamental Virology”, second edition, 1991, ed. B. N. Fields, Raven Press, New York, pages 489-503; and “Fields Virology”, third edition, 1995, ed. B. N. Fields, vol. 1, pages 1121-1159.

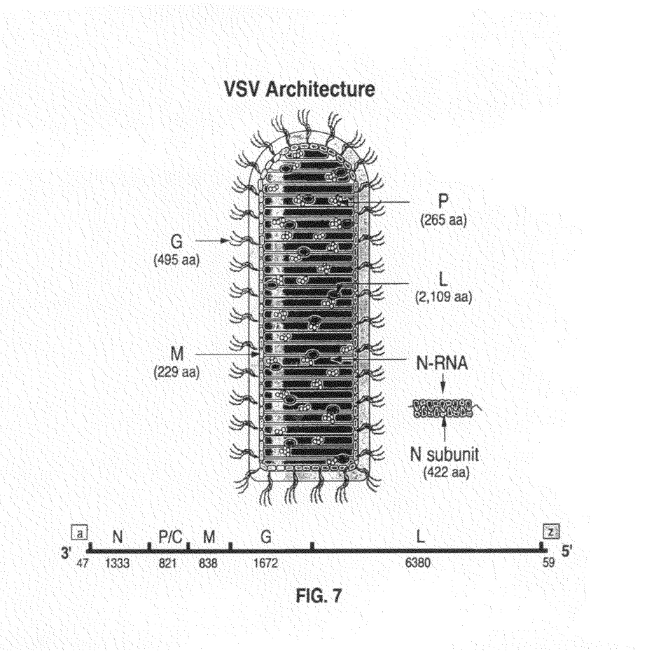

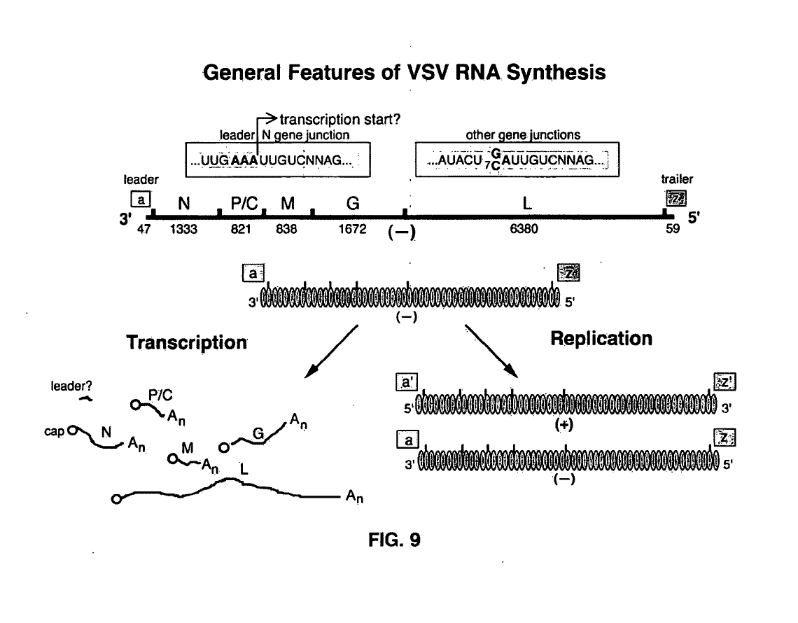

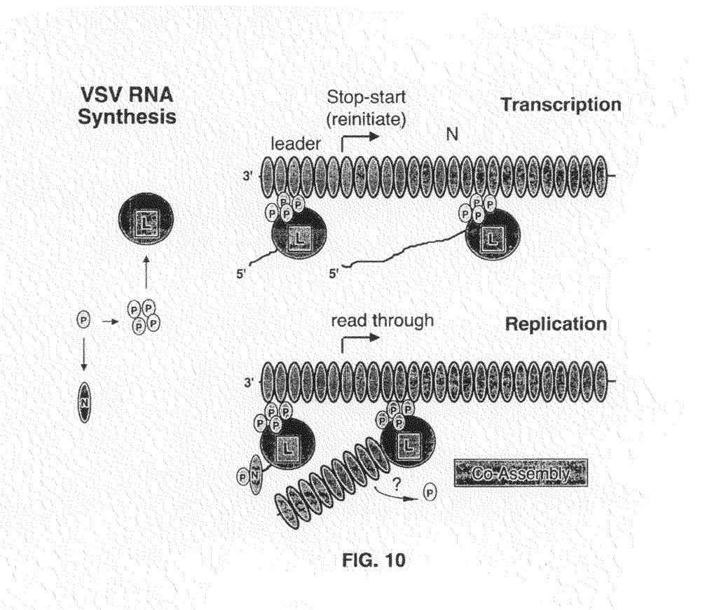

“VSV” as used herein refers to any strain of VSV or mutant forms of VSV, for example such as those described in WO 01/19380. A VSV construct of this invention may be in any of several forms, including, but not limited to, genomic RNA, mRNA, cDNA, part or all of the VSV RNA encapsulated in the nucleocapsid core, VSV complexed with compounds such as PEG and VSV conjugated to a nonviral protein. VSV vectors provided herein may encompass replication-competent and replication-defective VSV vectors, such as, VSV vectors lacking G glycoprotein of M glycoprotein. Replication-defective VSV vectors can be grown in appropriate cell lines. FIG. 7 represents a schematic of a vesicular stomatitis virus, including a representation of the VSV genome. General features of VSV RNA synthesis are illustrated in FIGS. 9 and 10.

a. VSV Sequences and Constructs

VSV, a member of the Rhabdoviridae family, is a negative-stranded virus that replicates in the cytoplasm of infected cells, does not undergo genetic recombination or reassortment, has no known transforming potential and does not integrate any part of it genome into the host. VSV comprises an about 11 kilobase genome that encodes for five proteins referred to as the nucleocapsid (N), polymerase proteins (L) and (P), surface glycoprotein (G) and a peripheral matrix protein (M). The genome is tightly encased in nucleocapsid (N) protein and also comprises the polymerase proteins (L) and (P). Following infection of the cell, the polymerase proteins initiate the transcription of five subgenomic viral mRNAs, from the negative-sense genome, that encode the viral proteins. The polymerase proteins are also responsible for the replication of the full-length viral genomes that are packaged into progeny virions. The matrix (M) protein binds to the RNA genome/nucleocapsid core (RNP) and also to the glycosylated (G) protein, which extends from the outer surface in an array of spike like projections and is responsible for binding to cell surface receptors and initiating the infectious process.

Following attachment of VSV through the (G) protein to receptor (s) on the host surface, the virus penetrates the host and uncoats to release the RNP particles. The polymerase proteins, which are carried in with the virus, bind to the 3′ end of the genome and sequentially synthesize the individual mRNAs encoding N, P, M, G, and L, followed by negative-sense progeny genomes. Newly synthesized N, P and L proteins associate in the cytoplasm and form RNP cores which bind to regions of the plasma membrane rich in both M and G proteins.

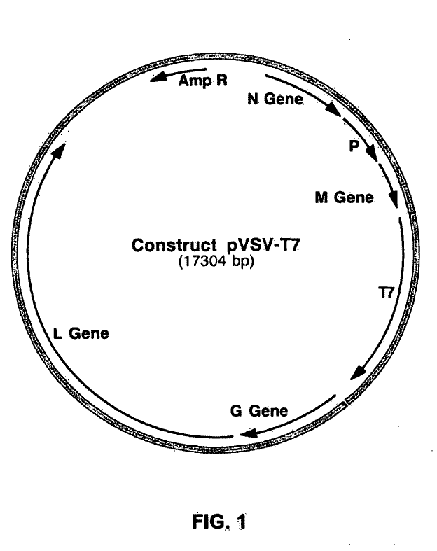

FIG. 1 depicts a schematic illustration of a VSV-T7 RNA polymerase plasmid as provided herein and as set forth in SEQ ID NO. 1.

b. Viral Particles Form and Budding or Release of Progeny Virus Ensues.

A table of various VSV strains is shown in “Fundamental Virology”, second edition, supra, at page 490. WO01/19380 and U.S. Pat. No. 6,168,943 disclose that strains of VSV include Indiana, New Jersey, Piry, Colorado, Coccal, Chandipura and San Juan. The complete nucleotide and deduced protein sequence of a VSV genome is known and is available as Genbank VSVCG, accession number J02428; NCBI Seq ID335873; and is published in Rose and Schubert, 1987, in The Viruses The Rhabdoviruses, Plenum Press, NY. pp. 129-166. A complete sequence of a VSV strain is shown in U.S. Pat. No. 6,168,943.

VSV New Jersey strain is available from the American Type Culture Collection (ATCC) and has ATCC accession number VR-159. VSV Indiana strain is available from the ATCC and has ATCC accession number VR-1421.

Provided herein are compositions and methods encompassing any form of VSV, including, but not limited to genomic RNA, mRNA, cDNA, and part or all of VSV RNA encapsulated in the nucleocapsid core. The present invention encompasses VSV in the form of a VSV vector construct as well as VSV in the form of viral particles. Also provided herein are nucleic acid encoding specific VSV vectors disclosed herein, such as set forth in SEQ ID NOs. 1 and 2. As provided herein, VSV vectors encompass replication-competent as well as replication-defective VSV vectors. Replication-competent VSV viral particles were prepared using standard methodology such as described by Whelan et al. (Proc Natl Acad Sci USA. 1995 Aug. 29; 92(18):8388-92.).

In certain aspects, the VSV vector lacks a protein function essential for assembly and release of infectious particles, such as G-protein function or M protein function. The VSV vector may lack several protein functions essential for replication. Such vectors are useful in producing VSV-T7 replication-defective viral particles. For example, plasmids encoding the M and G genes (pTM1-M and pTM1-G) can be co-transfected with standard plasmids encoding the L, P, and N proteins into vaccinia-T7 virus-infected BHK-21 cells. The co-transfected template plasmid in this case encodes the replication-defective virus genome, which includes the T7 RNA polymerase gene but not the M and G viral genes. Following initial packaging and release of the defective viral particles, further amplification of the defective particles is accomplished by co-transfection with pTM1-M and pTM1-G plasmids to supply the missing M and G proteins. In the absence of the complementing plasmids, replication-defective viruses replicate the defective genome and express N, P and L proteins in the infected cells, but the defective genomes are neither packaged nor released. Co-transfection of cells infected with VSV-T7 replication-defective viral vectors and with plasmids encoding a transgene leads to efficient protein expression of the transgene in the cell.

In other embodiments, heterologous surface proteins for VSV may come from any Rhabdovirus, and may also originate from a heterologous virus without modification, for example, influenza virus surface protein, or other viruses with some modifications, for example HIV. Replacement of the VSV Indiana G protein with that of VSV New Jersey serotype (no immune cross reaction) for second administration of a VSV vector in mice is known to those of ordinary skill in the art, such as, for example, work in Jack Rose's laboratory at Yale University.

In certain embodiments, the VSV G protein has been specifically engineered, by fusion with other proteins, to target desired cell types. (e.g., Gao et al., J. Virol. 80, 8603-12, 2006). Thus, by engineering the VSV vector, a broad range of different cells may be targeted.

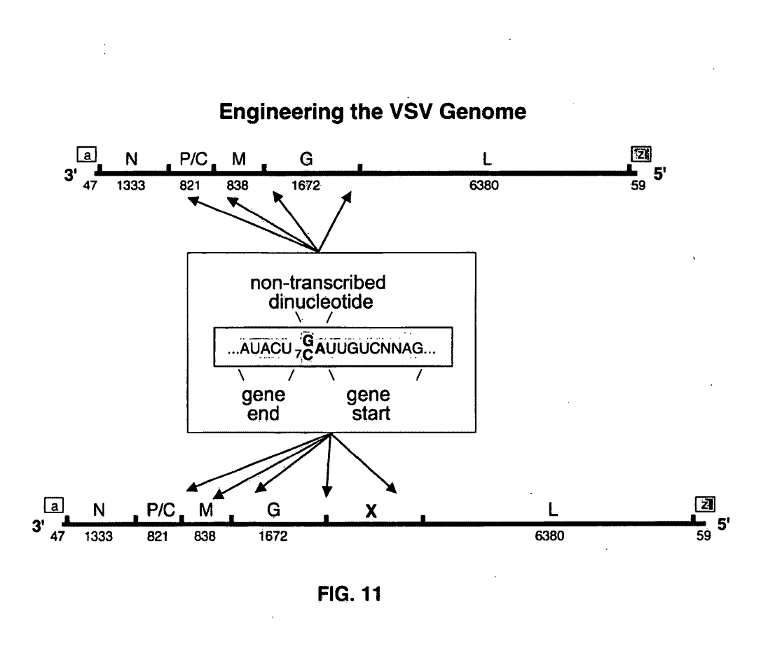

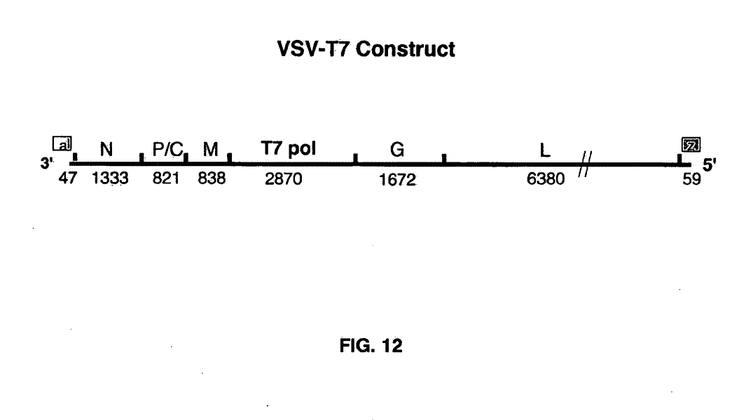

In certain embodiments, viral particles comprising a VSV vector provided herein encode the polynucleotide sequence for T7 RNA polymerase. Also provided is isolated nucleic acid encoding the recombinant VSV vector, as well as host cells comprising a recombinant VSV vector for producing such particles. Schematic illustrations of engineered VSV-T7 viral particle genomes are shown in FIGS. 11 and 12. Any foreign gene flanked by gene end and start signals are faithfully transcribed by the viral polymerase (see FIG. 11).

2. T7 RNA Polymerase Expression System

a. 1′7 RNA Polymerase and Promoter

As provided herein, the disclosed polypeptide expression system, involves infecting cultured cells with a VSV-T7 recombinant virus and transfecting these infected cells with a plasmid encoding a heterologous DNA sequence under control of the T7 promoter and an IRES element (see FIG. 12). The gene encoding T7 RNA polymerase is derived from a prokaryotic source. In one aspect, the VSV-T7 recombinant virus vector is engineered to express a prokaryotic T7 RNA polymerase enzyme in the cytoplasm of infected cells. The infected cells are then transfected with a recombinant plasmid vector encoding a heterologous DNA downstream of a T7 promoter sequence and an IRES element. This results in cytoplasmic accumulation of large amounts of T7 mRNA transcripts which are efficiently translated into the desired protein.

In another aspect, then RNA polymerase enzyme in the cytoplasm of infected cells is capable of transcription of a heterologous DNA downstream of a T7 promoter sequence, but without an IRES element. In such circumstances, the cell is co-transfected with plasmids expressing the two subunits of the vaccinia virus-capping enzyme under control of the virus, as discussed in detail below.

As provided herein, a T7 promoter sequence facilitates binding of T7 polymerase to a polynucleotide sequence for the initiation of transcription. Also contemplated are any bacteriophage promoter sequences that can be recognized by an RNA polymerase that is expressesd in the host.

b. IRES

T7 transcripts synthesized using the VSV-T7 expression system provided herein lack cap structures at their 5′ ends and thus require an IRES element for efficient translation. The IRES element is capable of providing cap-independent translation of a downstream gene or coding sequence by an internal ribosome entry mechanism. In certain aspects, recombinant plasmid vectors provided herein encode a T7 promoter sequence, an IRES element and a heterologous polynucleotide sequence for efficient transcription and expression of the desired protein.

c. Vaccinia Virus-Capping Enzymes

To circumvent the requirement for an IRES element in the plasmid encoding the heterologous polynucleotide of interest provided herein are compositions and methods for co-transfecting target cells with plasmid vectors encoding the D1 and D12 subunits of vaccinia virus-capping enzyme, as set forth in SEQ ID NOs. 5 and 6. Expression of the vaccinia virus-capping enzyme results in capping of the 5′ end of nascent transcripts, thereby facilitating efficient translation. The capping enzyme sequences may be provided as separate plasmid vectors.

3. Recombinant Plasmids

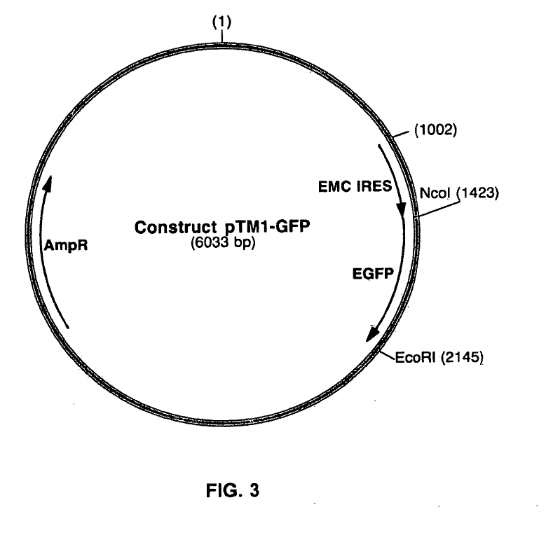

Recombinant plasmids provided herein were constructed using standard molecular biology cloning techniques known to those of ordinary skill in the art. Recombinant VSV viruses that express T7 RNA polymerase can drive expression of the desired heterologous polynucleotide sequence encoded on plasmids under control of a T7 promoter. Other bacteriophage promoter sequences may also be used. FIG. 3 illustrates plasmid pTM1-GFP (SEQ ID NO. 3) encoding an IRES element and the green fluorescent protein (GFP) reporter gene. The GFP gene from pBI-GFP was inserted into the NcoI site of the pTM-1 vector using standard restriction enzyme cloning techniques. As will be recognized by one of skill in the art, the GFP gene can be replaced by any heterologous polynucleotide sequence using appropriate restriction enzyme recognition sites and standard molecular biology techniques.

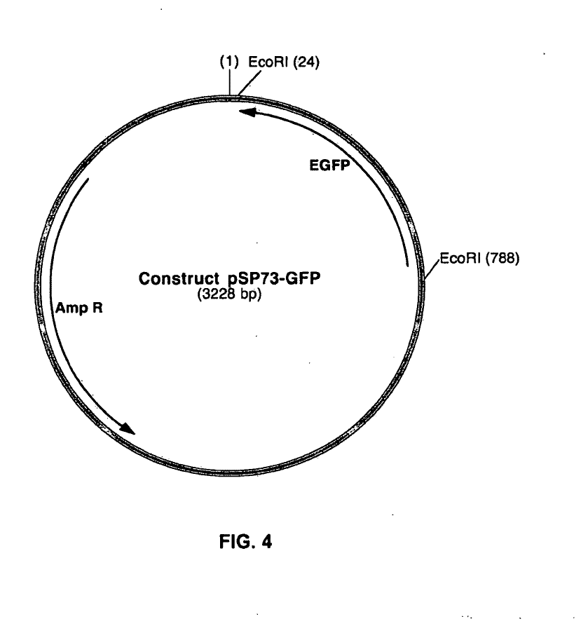

FIG. 4 illustrates plasmid pSP73-GFP (SEQ ID NO. 4) encoding the GFP reporter gene, but without an IRES element. The GFP gene from pBI-EGFP vector was inserted into the EcoRI site of the pSP73 vector. As noted above, the GFP can be replaced by any heterologous nucleotide sequence using standard restriction enzyme cloning techniques.

4. Expression of Polypeptides

As described herein, the VSV-T7 expression system is used to infect a cell with VSV-T7 viral vector particles followed by transfection of recombinant plasmids encoding a transgene using techniques well known to those of skill in the art (see, e.g., Wigler et al. (1979) Proc. Natl. Acad. Sci. USA 76:1373-1376; Cohen et al. (1972) Proc. Natl. Acad. Sci. USA 69:2110). One embodiment as described herein, involves infecting cultured cells with a VSV-T7 recombinant virus and transfecting these infected cells with a plasmid encoding a heterologous polynucleotide sequence (e.g. transgene) under control of the T7 promoter and an IRES element. Although VSV normally shuts off host cell protein synthesis, it was found that T7 transcripts are efficiently translated under these conditions to yield protein amounts comparable to the vaccinia-T7 system. T7 transcripts synthesized in this VSV-T7 system lack cap structures at their 5′ end and thus require an IRES element for translation. In another embodiment provided herein, compositions and methods are provided wherein VSV-T7 infected cells are co-transfected with plasmids encoding the two subunits of the vaccinia virus-capping enyzme (SEQ ID NOs. 5 and 6) under control of an IRES element, thereby circumventing the requirement for an IRES element in the plasmid encoding the transgene of interest. Also contemplated is a defective VSV-T7 recombinant virus that lacks virus-encoded host cell shutoff functions for improving expression of the transgene.

4. Expression of Interfering RNAs

Provided herein is a viral vector expression system for delivery of a nucleic acid sequence encoding a small interfering RNA molecule (siRNA) targeted against a gene of interest.

The target gene may be a gene derived from the cell, an endogenous gene, a transgene, or a gene of a pathogen which is present in the cell after infection thereof. Depending on the particular target gene and the dose of double stranded RNA material delivered, the procedure may provide partial or complete loss of function for the target gene. A reduction or loss of gene expression in at least 99% of targeted cells has been shown. Lower doses of injected material and longer times after administration of dsRNA may result in inhibition in a smaller fraction of cells. Quantitation of gene expression in a cell may show similar amounts of inhibition at the level of accumulation of target mRNA or translation of target protein.

The RNA may comprise one or more strands of polymerized ribonucleotide. The double-stranded structure may be formed by a single self-complementary RNA strand, by two complementary RNA strands or by co-transfecting cells with plasmids encoding transgenes or fragments thereof in opposite orientation relative to the T7 promoter. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequence identical to a portion of the target gene can be used for inhibition. RNA sequences with insertions, deletions, and single point mutations relative to the target sequence have also been found to be effective for inhibition. Thus, sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

The cell with the target gene may be derived from or contained in any organism (e.g., plant, animal, protozoan, virus, bacterium, or fungus). RNA may be synthesized either in vivo or in vitro. Cloned RNA polymerase as provided by the VSV-T7 expression system may mediate transcription in vivo. For transcription from a transgene in vivo or an expression construct, a regulatory region, i.e., the T7 promoter, may be used to transcribe the RNA strand (or strands) (the regulatory sequence is the T7 promoter). A method of reducing the expression of a gene product in a cell, comprising contacting a cell with the VSV viral particle encoding T7 RNA polymerase and appropriate recombinant plasmid vector encoding the desired interfering RNA nucleotide sequence are also provided

RNA interference is now established as an important biological strategy for gene silencing, but its application to mammalian cells has been limited by nonspecific inhibitory effects of long double-stranded RNA on translation. Provided herein are compositions and methods for a viral mediated delivery mechanism that results in delivery of small interfering RNA (siRNA) into a target cell. This viral mediated strategy is generally useful in reducing expression of target genes in order to model biological processes or to provide therapy for dominant human diseases. Those of ordinary skill in the art will recognize that the use of a substantially attenuated VSV strain, that is, for example, capable of infecting a cell, yet allowing the cell to survive for longer periods of time when compared to wild type or a less attenuated VSV vector, would be advantageous for the delivery of siRNA.

### C. Formulation and Administration

While the compositions and methods of the present invention will typically be used in therapy for human patients, they may also be used in veterinary medicine. The compositions may, for example, be used to treat mammals, including, but not limited to, primates and domesticated mammals. The compositions may, for example be used to treat herbivores.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

The exact dosage will depend upon the route of administration, the form in which the composition is administered, the subject to be treated, the age, body weight/height of the subject to be treated, and the preference and experience of the attending physician.

The compositions of the present invention may include pharmaceutically acceptable salts. Pharmaceutically acceptable salts are generally well known to those of ordinary skill in the art and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20.sup.th ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Administration of pharmaceutically acceptable salts of the DNA molecules described herein is included within the scope of the invention. For example, pharmaceutically acceptable salts may be prepared from non-toxic bases including organic bases and inorganic bases. Salts derived from inorganic bases include sodium, potassium, lithium, ammonium, calcium, magnesium, and the like. Salts derived from pharmaceutically acceptable organic nontoxic bases include salts of primary, secondary, and tertiary amines, basic amino acids, and the like. Pharmaceutically acceptable salts may be found in, for example, S. M. Berge et al., Journal of Pharmaceutical Sciences 66:1-19 (1977).

Methods of delivering DNA vaccines, as well as formulations and methods of administration may be found in, for example, U.S. Pat. Nos. 6,806,084 and 5,580,859. The DNA vaccine may, for example, be formulated to include transfection-facilitating proteins, viral particles, liposomal formulations, charged lipids and calcium phosphate precipitating agents, or it may not include these components. Those of ordinary skill in the art may determine the appropriate formulation, considering factors such as the route of administration, for example, intradermal, intramuscular, or intranasal. Methods of delivering vesicular stomatitis vectors are known to those of ordinary skill in the art and are exemplified in Cooper, D. et al., J. Virol. 2007 (Oct. 17, 2007) (e-publication ahead of print, found at http://jvi.asm.org (J. Virol. Doi: 10.1128/JVI.01515-07, which discusses, for example, a mouse model for VSV-mediated vaccination and assessment.

In addition to the active ingredients, these pharmaceutical compositions may contain suitable pharmaceutically acceptable carriers comprising excipients and auxiliaries.

### D. Kits

The present invention further provides kits comprising vaccine compositions or components that may be used to prepare vaccine compositions. For example, such kits may comprise systems, the components of said system may be, for example, formulated for vaccine delivery. Such kits may further comprise a second vaccine composition, comprising, for example, a chemically inactivated virus. Or, for example, such kits may provide chemicals that may be used to inactivate viruses, such as, for example formalin; such kits may further comprise virus that has not yet been chemically inactivated.

Kits may also include instructions, and other components needed for immunization, such as, for example, nasal, muscular, or dermal delivery systems, such as, for example, needles, syringes, and inhalation or misting devices.

### EXAMPLES

The following examples are provided to illustrate certain embodiments of the invention and are not limiting. Where the examples refer to T7 expression systems, it is to be understood by those of ordinary skill in the art that these systems can be readily adapted for other bacteriophage RNA polymerase/bacteriophage promoter systems, such as, for example, T7, Sp6, T1, T3, T5 and the like.

### Example 1

### Vaccinia-T7 Expression System

High level expression of a reporter protein (green fluorescent protein) using an IRES-containing plasmid construct was accomplished using the VSV-T7 expression system provided herein and shown to be comparable in efficiency to the vaccinia-T7 system. Infectious VSV-T7 viral particles were recovered from BHK-21 cells (baby hamster kidneys) transfected with pVSV-T7 (SEQ ID NO. 1; FIG. 1) grown in minimal essential medium (MEM) supplemented with 7% newborn calf serum using standard tissue culture techniques. Supernatants containing viral particles were titered by plaque assay and used as inoculae for expression studies. Vaccinia-T7 virus stocks were prepared using similar techniques in BSC-40 cells (monkey kidney). All expression experiments were performed using nearly confluent BHK-21 cells grown in monolayers in 5 cm plates (˜1×10⁶ cells per plate). All virus adsorptions were performed at a multiplicity of 10 pfu/cell in a volume of 0.3 ml for 1 hour at room temperature. Vaccinia-T7 inoculae also included 10 micrograms/ml of DEAE-dextran and 40 micrograms/ml Ara-C.

Following adsorption, the virus inoculae were removed before addition of 0.5 ml of the transfection reagents containing 45 microliters lipofection reagent (prepared as described by Rose et al. (Biotechniques 1991, 10:520-525)) and 455 microliters of MEM containing various amounts of plasmid encoding the reporter gene. After 1 hr, 1.5 ml of fresh MEM plus 7% newborn calf serum was added (containing 40 micrograms/ml Ara-C for vaccinia-T7 infections) and monolayers were incubated at 37° C. Expression of GFP was examined by fluorescent microscopy at various times.

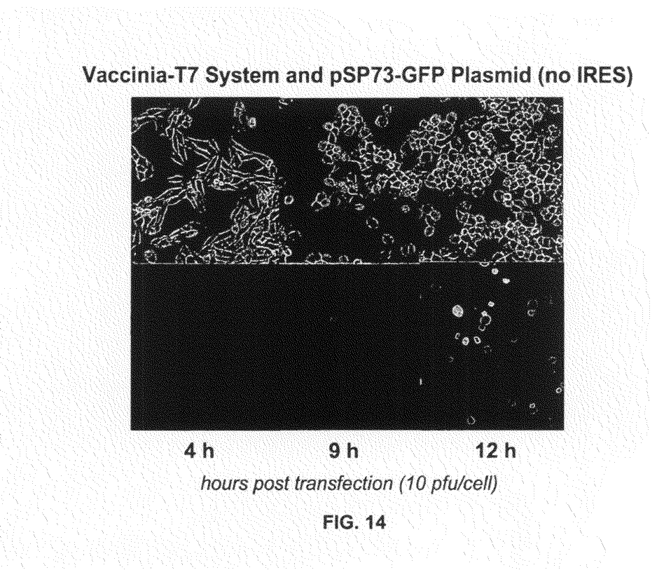

FIG. 13 shows expression of GFP in cells infected with vaccinia-T7 viral particles, followed by transfection with 11 micrograms of pTM1-GFP (with IRES), at 4 h, 9 h, and 12 h, post-transfection (bottom panel). Cells infected with vaccinia-T7 viral particles and then transfected with 11 micrograms of pSP73-GFP (no IRES) are shown in FIG. 14 (GFP expressing cells shown in the bottom panel). The level of GFP expression is similar with or without an IRES element encoded in the expression plasmid when using a vaccinia-T7 viral expression system.

### Example 2

### VSV-T7 Expression System

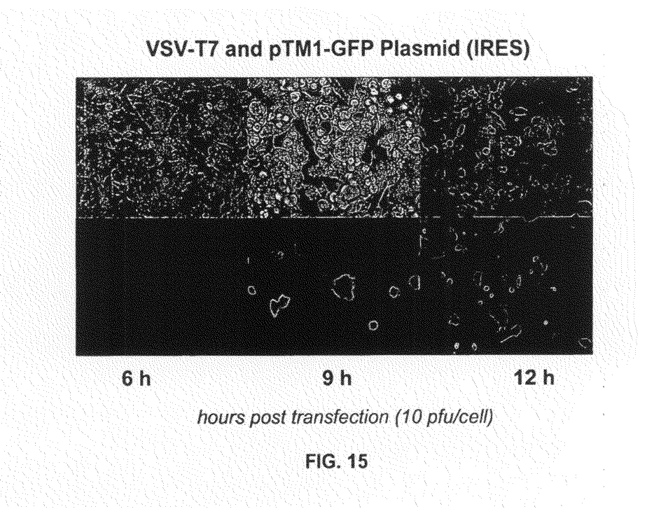

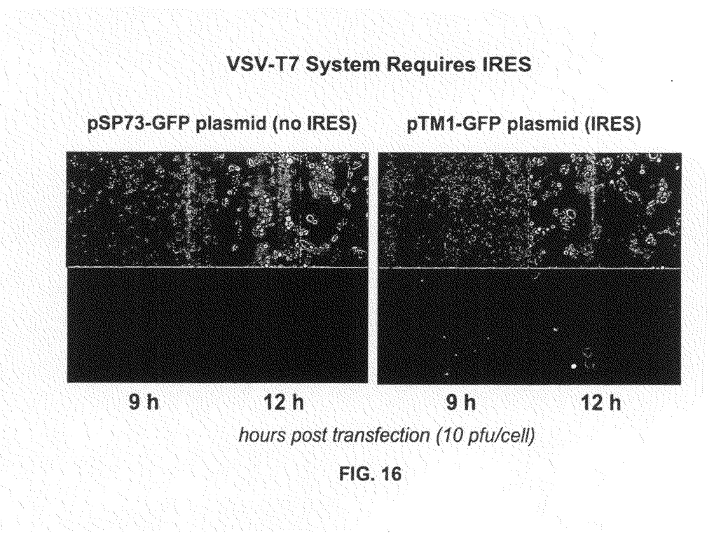

FIG. 15 shows GFP expression in cells infected with VSV-T7 viral particles and transfected with 11 micrograms of pTM1-GFP (with IRES) at 6 h, 9 h and 12 h post-transfection (bottom panel). Expression of the same reporter gene without an IRES element, as shown in FIG. 16, indicates that the VSV-T7 system requires an IRES element for efficient expression of the transgene if 5′ capping enzymes are not provided.

### Example 3

### Comparison of Vaccinia-T7 and VSV-T7 Expression Systems

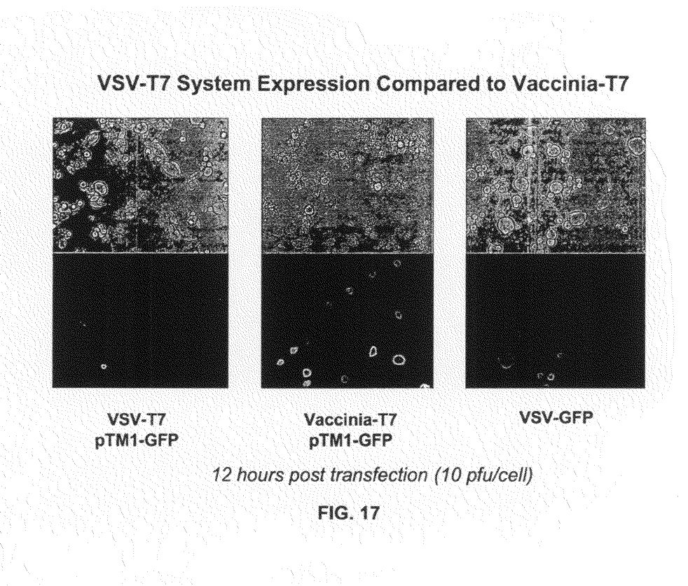

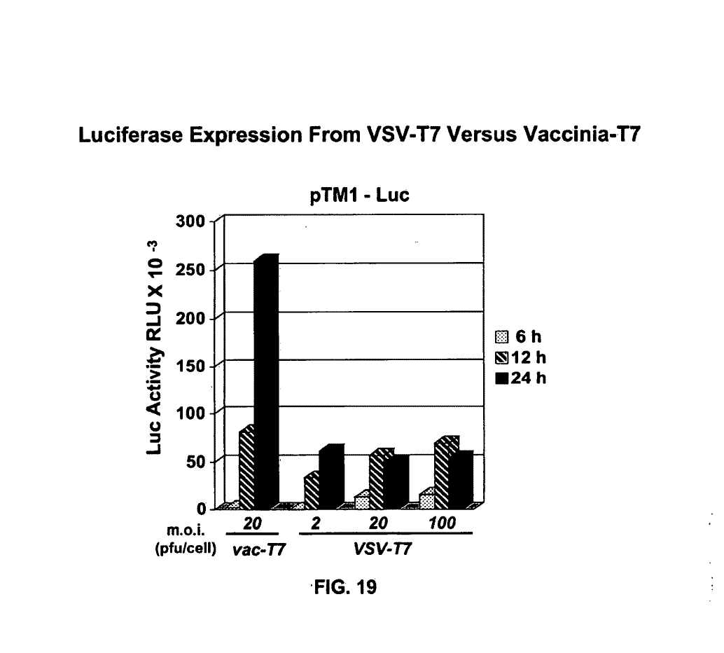

A comparison of the VSV-T7 expression system with the vaccinia-T7 system indicates similar levels of reporter gene expression at 12 hours post-infection when cells are transfected with plasmids encoding an IRES (FIG. 17, bottom panel). As a comparison, cells infected with VSV viral particles encoding GFP instead of T7 RNA polymerase also express GFP at comparable levels at 12 hours post-infraction (far right panels). The vaccinia T7 and VSV-T7 systems also produced similar levels of the reporter firefly luciferase gene, following essentially the same methods, at 12 hours post infection, when the luciferase gene was used in place of GFP in the pTM1 plasmid (FIG. 19).

### Example 4

### VSV-T7 Expression System with Vaccinia Virus-Capping Enzymes

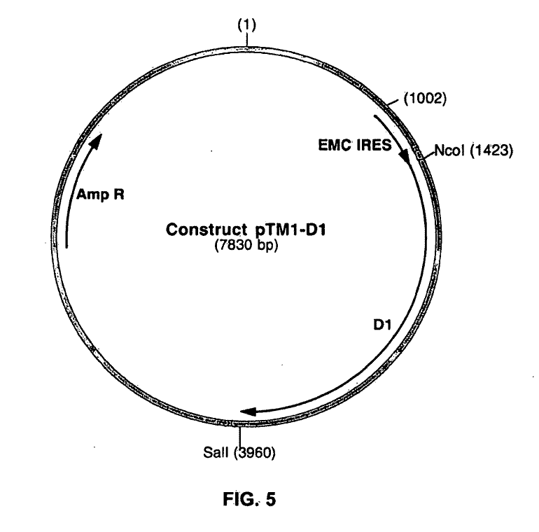

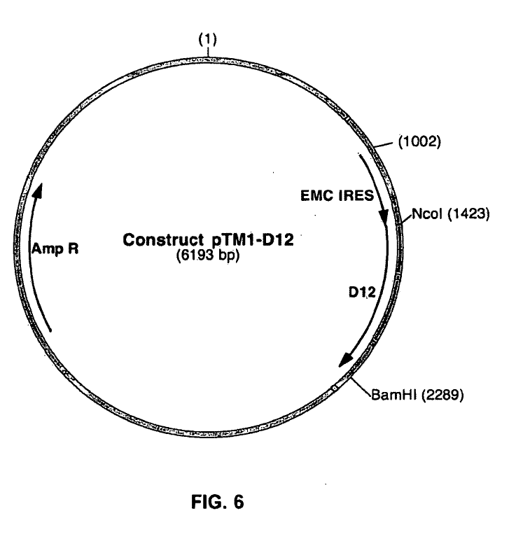

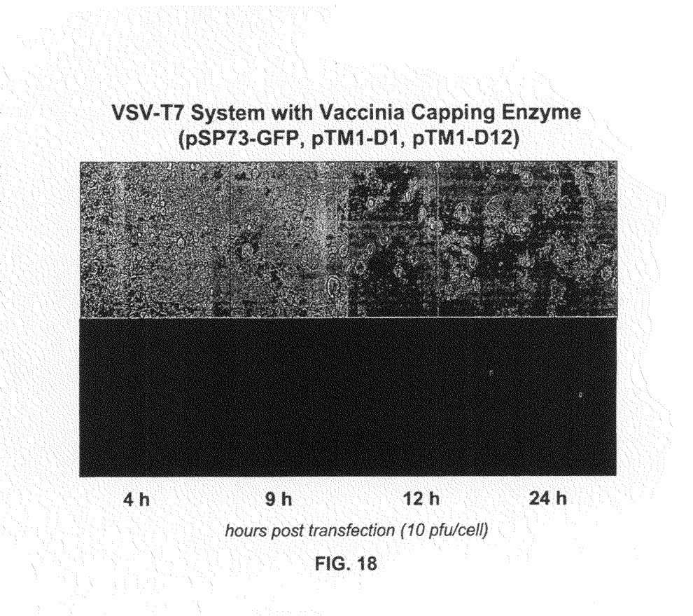

To circumvent the requirement of an IRES element in the plasmid driving expression of the transgene, cells were co-transfected with plasmids encoding the D1 and D12 subunits of the vaccinia virus-capping enzyme under control of an IRES element (SEQ ID NOs. 5 and 6; FIGS. 5 and 6). Capping functions at the 5′ end of the transcript are therefore provided in trans, thus bypassing the need for an IRES element. GFP expression of cells infected with VSV-T7 viral particles followed by co-transfection with pSP73-GFP (no IRES), pTM1D1 and pTM1-D12 plasmids is shown in FIG. 18. These results indicate that the VSV-T7 expression system can be used successfully without the requirement of an IRES element if the transgene 5′ capping functions are provided in trans.

### Example 5

### Examples of Embodiments

Provided hereafter is a listing of certain non-limiting embodiments of the invention.

A1. A method for producing a heterologous protein in a cell, comprising contacting said cell with
a) a recombinant plasmid vector comprising a polynucleotide comprising the following elements operably linked 5′ to 3′: (i) a bacteriophage promoter sequence, and (ii) a heterologous gene; and
b) a vesicular stomatitis virus vector particle (VSV) comprising a polynucleotide encoding a bacteriophage RNA polymerase that operates on said bacteriophage promoter sequence.
   A2. The method of embodiment A1, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   A3. The method of any of embodiments A1-A2, wherein said plasmid vector is circular.
   A4. The method of any of embodiments A1-A2, wherein said plasmid vector is linear.
   A5. The method of any of embodiments A1-A3, wherein said plasmid comprises chemically modified DNA.
   A6. The method of any of embodiments A1-A5, wherein said heterologous gene comprises a sequence encoding an internal ribosome entry site.
   A7. The method of any of embodiments A1-A6, further comprising contacting said cell with a DNA sequence encoding a vaccinia capping enzyme.
   A8. The method of embodiment A7, comprising contacting said cell with a first DNA sequence encoding the D1 catalytic subunit of vaccinia capping enzyme, and a second DNA sequence encoding the D12 subunit of vaccinia capping enzyme.
   A9. The method of embodiment A8, comprising contacting said cell with a recombinant plasmid capping vector comprising said first DNA sequence and said second DNA sequence.
   A10. The method of embodiment A8, comprising contacting said cell with a first recombinant plasmid capping vector comprising said first DNA sequence and with a second recombinant plasmid capping vector comprising said second DNA sequence.
   A11. The method of any of embodiments A8-A10, wherein said first DNA sequence comprises the sequence set forth in SEQ NO. 5 and said second DNA sequence comprises the sequence set forth in SEQ ID NO. 6.
   A12. The method of any of embodiments A1-11, wherein said bacteriophage promoter sequence is operably linked to a bacteriophage gene expression regulator sequence.
   A13. The method of embodiment A12, wherein said regulator sequence is selected from the group consisting of a ribo-switch sequence and a ligand-regulated protein binding site.
   A14. The method of any of embodiments A1-13, further comprising contacting said cell with a DNA sequence encoding a protein that regulates bacteriophage RNA polymerase activity.
   A15. The method of embodiment A14, wherein said DNA sequence encodes a lysozyme.
   A16. The method of embodiment A14 or A15, wherein the recombinant plasmid vector of embodiment A1 comprises said DNA sequence encoding the protein that regulates bacteriophage RNA polymerase activity.
   A17. The method of embodiment A14 or A15, comprising contacting said cell with a recombinant plasmid RNA polymerase regulator vector comprising said DNA sequence encoding the protein that regulates bacteriophage RNA polymerase activity.
   A18. The method of any of embodiments A1-A17, further comprising contacting said cell with a DNA sequence encoding a protein that modulates the immune response of a host animal.
   A19. The method of embodiment A18, wherein said host animal is a human.
   A20. The method of embodiment A18, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   A21. The method of embodiment A20, wherein said enhancer of antigen presentation to APCs is GM-CSF.
   A22. The method of embodiment A20, wherein said factor that helps recruit and activate DCs is MIP-1α or Flt3L.
   A23. The method of embodiment A20, wherein said enhancer of T-lymphocyte priming is B7-2 or CD154.
   A24. The method of embodiment A20, wherein said stimulator of T-lymphocyte expansion is selected from the group consisting of IL-2, IL-12, and IL-15.
   A25. The method of embodiment A18, wherein the recombinant plasmid vector of embodiment A1 comprises said DNA sequence encoding the protein that modulates the immune response of a host animal.
   A26. The method of embodiment A18, comprising contacting said cell with a recombinant plasmid immune modulating vector comprising said DNA sequence encoding the protein that modulates the immune response of a host animal.
   A27. The method of any of embodiments A1-A26, wherein said recombinant plasmid vector comprises a T7 promoter corresponding to residues 794 to 813 of SEQ ID No. 3.
   A28. The method of any of embodiments A1-A27, wherein said vesicular stomatitis virus vector particle comprises the polynucleotide sequence of SEQ ID No. 1.
   B1. A method for producing two or more heterologous proteins in a cell, comprising contacting said cell with
a) a recombinant plasmid vector comprising a polynucleotide comprising the following elements operably linked 5′ to 3′: (i) a bacteriophage promoter sequence, and (ii) a DNA sequence encoding two or more heterologous proteins; and
b) a vesicular stomatitis virus vector particle (VSV) comprising a polynucleotide encoding a bacteriophage RNA polymerase that operates on said bacteriophage promoter sequence.
   B2. The method of embodiment B1, wherein said DNA sequence encoding two or more heterologous proteins comprises two or more sequences encoding internal ribosome entry sites, each of which enables translation of a different heterologous protein.
   B3. The method of embodiment B1, wherein said heterologous gene encodes a fusion protein comprising two or more heterologous proteins.
   B4. The method of embodiment B1, wherein said plasmid comprises two or more bacteriophage promoter sequences, and said cell is contacted with a DNA sequence encoding a DNA endonuclease, said DNA endonuclease is expressed in said cell, and said DNA endonuclease releases individual expression cassettes from said plasmid, said expression cassettes comprising a bacteriophage promoter sequence and a DNA sequence encoding a heterologous protein.
   B5. The method of embodiment B4, wherein said recombinant plasmid comprises said DNA sequence encoding a DNA endonuclease.
   B6. The method of embodiment B5, comprising contacting said cell with a second recombinant plasmid vector, wherein said second recombinant plasmid vector comprises a DNA sequence encoding a DNA endonuclease.
   B7. The method of embodiment B1, further comprising contacting said cell with a DNA sequence encoding a ribozyme, said ribozyme is expressed in said cell, wherein said ribozyme cleaves transcripts encoding said two or more heterologous proteins to provide two or more individual transcripts, each of which codes for a different heterologous protein.
   B8. The method of embodiment B7, wherein each of said individual transcripts comprises an internal ribosome entry sequence.
   B9. The method of any of embodiments B1-B8, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   B10. The method of any of embodiments B1-B9, wherein said plasmid vector is circular.
   B11. The method of any of embodiments B1-B9, wherein said plasmid vector is linear.
   B12. The method of any of embodiments B1-B11, wherein said plasmid comprises chemically modified DNA.
   B13. The method of any of embodiments B1-B11, wherein three or more heterologous proteins are expressed in said cell.
   B14. The method of any of embodiments B1-B11, wherein four or more heterologous proteins are expressed in said cell.
   B15. The method of any of embodiments B1-B11, wherein five or more heterologous proteins are expressed in said cell.
   B16. The method of any of embodiments B1-B15, further comprising contacting said cell with a DNA sequence encoding a vaccinia capping enzyme.
   B17. The method of embodiment B16, comprising contacting said cell with a first DNA sequence encoding the D1 catalytic subunit of vaccinia capping enzyme, and a second DNA sequence encoding the D12 subunit of vaccinia capping enzyme.
   B18. The method of embodiment B17, comprising contacting said cell with a recombinant plasmid capping vector comprising said first DNA sequence and said second DNA sequence.
   B19. The method of embodiment B17, comprising contacting said cell with a first recombinant plasmid capping vector comprising said first DNA sequence and with a second recombinant plasmid capping vector comprising said second DNA sequence.
   B20. The method of any of embodiments B17-B19, wherein said first DNA sequence comprises the sequence set forth in SEQ ID NO. 5 and said second DNA sequence comprises the sequence set forth in SEQ ID NO. 6.
   B21. The method of any of embodiments B1-B20, wherein said bacteriophage promoter sequence is operably linked to a bacteriophage gene expression regulator sequence.
   B23. The method of embodiment B21, wherein said regulator sequence is selected from the group consisting of a ribo-switch sequence and a ligand-regulated protein binding site.
   B24. The method of any of embodiments B1-B23, further comprising contacting said cell with a DNA sequence encoding a protein that regulates bacteriophage RNA polymerase activity.
   B25. The method of embodiment B24, wherein said DNA sequence encodes a lysozyme.
   B26. The method of embodiment B24 or B25, wherein the recombinant plasmid vector of embodiment B1 comprises said DNA sequence encoding the protein that regulates bacteriophage RNA polymerase activity.
   B27. The method of embodiment B24 or B25, comprising contacting said cell with a recombinant plasmid RNA polymerase regulator vector comprising said DNA sequence encoding the protein that regulates bacteriophage RNA polymerase activity.
   B28. The method of any of embodiments B1-B27, further comprising contacting said cell with a DNA sequence encoding a protein that modulates the immune response of a host animal.
   B29. The method of embodiment B28, wherein said host animal is a human.
   B30. The method of embodiment B28, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   B31. The method of embodiment B30, wherein said enhancer of antigen presentation to APCs is GM-CSF.
   B32. The method of embodiment B30, wherein said factor that helps recruit and activate DCs is MIP-1α or FIt3L.
   B33. The method of embodiment B30, wherein said enhancer of T-lymphocyte priming is B7-2 or CD154.
   B34. The method of embodiment B30, wherein said stimulator of T-lymphocyte expansion is selected from the group consisting of IL-2, IL-12, and IL-15.
   B35. The method of embodiment B28, wherein the recombinant plasmid vector of embodiment B1 comprises said DNA sequence encoding the protein that modulates the immune response of a host animal.
   B36. The method of embodiment B28, comprising contacting said cell with a recombinant plasmid immune modulating vector comprising said DNA sequence encoding the protein that modulates the immune response of a host animal.
   B37. The method of any of embodiments B1-B36, wherein said recombinant plasmid vector comprises a T7 promoter corresponding to residues 794 to 813 of SEQ ID No. 3.
   B38. The method of any of embodiments B1-B37, wherein said vesicular stomatitis virus vector particle comprises the polynucleotide sequence of SEQ ID No. 1.

C1. A method for producing two or more heterologous proteins in a cell, comprising contacting said cell with
a) two or more recombinant plasmid vectors, each comprising a polynucleotide comprising the following elements operably linked 5′ to 3′: (i) a bacteriophage promoter sequence, and (ii) a DNA sequence encoding a heterologous protein; and
b) a vesicular stomatitis virus vector particle (VSV) comprising a polynucleotide encoding a bacteriophage RNA polymerase that operates on said bacteriophage promoter sequence.
   C2. The method of embodiment C1, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   C3. The method of any of embodiments C1-C2, wherein said plasmid vectors are circular.
   C4. The method of any of embodiments C1-C2, wherein said plasmid vectors are linear.
   C5. The method of any of embodiments C1-C3, wherein said plasmid vectors comprise chemically modified DNA.
   C6. The method of any of embodiments C1-C5, wherein said heterologous genes comprise a sequence encoding an internal ribosome entry site.
   C7. The method of any of embodiments C1-C6, wherein three or more heterologous proteins are expressed in said cell.
   C8. The method of any of embodiments C1-C6, wherein four or more heterologous proteins are expressed in said cell.
   C9. The method of any of embodiments C1-C6, wherein five or more heterologous proteins are expressed in said cell.
   C10. The method of any of embodiments C1-C6, wherein six or more heterologous proteins are expressed in said cell.
   C11. The method of any of embodiments C1-C6, wherein seven or more heterologous proteins are expressed in said cell.
   C12. The method of any of embodiments C1-C6, wherein eight or more heterologous proteins are expressed in said cell.
   C13. The method of any of embodiments C1-C12, further comprising contacting said cell with a DNA sequence encoding a vaccinia capping enzyme.
   C14. The method of embodiment C13, comprising contacting said cell with a first DNA sequence encoding the D1 catalytic subunit of vaccinia capping enzyme, and a second DNA sequence encoding the D12 subunit of vaccinia capping enzyme.
   C15. The method of embodiment C14, comprising contacting said cell with a recombinant plasmid capping vector comprising said fust DNA sequence and said second DNA sequence.
   C16. The method of embodiment C14, comprising contacting said cell with a first recombinant plasmid capping vector comprising said first DNA sequence and with a second recombinant plasmid capping vector comprising said second DNA sequence.
   C17. The method of any of embodiments C14-C16, wherein said first DNA sequence comprises the sequence set forth in SEQ ID NO. 5 and said second DNA sequence comprises the sequence set forth in SEQ ID NO. 6.
   C18. The method of any of embodiments C1-C17, wherein said bacteriophage promoter sequence is operably linked to a bacteriophage gene expression regulator sequence.
   C19. The method of embodiment C18, wherein said regulator sequence is selected from the group consisting of a ribo-switch sequence and a ligand-regulated protein binding site.
   C20. The method of any of embodiments C1-C19, further comprising contacting said cell with a DNA sequence encoding a protein that regulates bacteriophage RNA polymerase activity.
   C21. The method of embodiment C20, wherein said DNA sequence encodes a lysozyme.
   C22. The method of embodiment C20 or C21, wherein at least one recombinant plasmid vector of embodiment C1 comprises said DNA sequence encoding the protein that regulates bacteriophage RNA polymerase activity.
   C23. The method of embodiment C20 or C21, comprising contacting said cell with a recombinant plasmid RNA polymerase regulator vector comprising said DNA sequence encoding the protein that regulates bacteriophage RNA polymerase activity.
   C24. The method of any of embodiments C1-C23, further comprising contacting said cell with a DNA sequence encoding a protein that modulates the immune response of a host animal.
   C25. The method of embodiment C24, wherein said host animal is a human.
   C26. The method of embodiment C24, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   C27. The method of embodiment C26, wherein said enhancer of antigen presentation to APCs is GM-CSF.
   C28. The method of embodiment C26, wherein said factor that helps recruit and activate DCs is MIP-1α or FIt3L.
   C29. The method of embodiment C26, wherein said enhancer of T-lymphocyte priming is B7-2 or CD154.
   C30. The method of embodiment C26, wherein said stimulator of T-lymphocyte expansion is selected from the group consisting of IL-2, IL-12, and IL-15.
   C31. The method of embodiment C24, wherein at least one recombinant plasmid vector of embodiment C1 comprises said DNA sequence encoding the protein that modulates the immune response of a host animal.
   C32. The method of embodiment C24, comprising contacting said cell with a recombinant plasmid immune modulating vector comprising said DNA sequence encoding the protein that modulates the immune response of a host animal.
   C33. The method of any of embodiments C1-C32, wherein at lease one of said recombinant plasmid vectors comprises a T7 promoter corresponding to residues 794 to 813 of SEQ ID No. 3.
   C34. The method of any of embodiments C1-C33, wherein said vesicular stomatitis virus vector particle comprises the polynucleotide sequence of SEQ ID No. 1.
   D1. The method of any of embodiments A1-A28, B1-B38, or C1-C34, wherein cells are contacted with said recombinant plasmid vector by injection into a host.
   D2. The method of any of embodiments A1-A28, B1-B38, or C1-C34, wherein cells are contacted with said recombinant plasmid vector with an agent that promotes entry of DNA into cells.
   D3. The method of embodiment D3, wherein said agent is selected from the group consisting of lipids, polymers, and gold particles.
   D4. The method of any of embodiments A1-A28, B1-B38, or C1-C34, wherein cells are contacted with said recombinant plasmid vector by a method selected from the group consisting of liposome mediated transfer, lipofection, polycation-mediated transfer, direct DNA transfer, electroporation, gene gun use, transfection in the presence of polymers, and transfection in the presence of gold particles.
   D5. The method of any of embodiments A1-A28, B1-B38, or C1-C34, wherein said cells are contacted with said recombinant plasmid vector by administration to an animal.
   D6. The method of embodiment D5, wherein said animal is a mammal.
   D7. The method of embodiment D6, wherein said mammal is a human.
   D8. The method of embodiment D5, wherein said administration is by a method selected from the group consisting of intra-muscular injection, intra-peritoneal injection, intravenous delivery, subcutaneous deliver, intra-nasal delivery, and oral delivery.
   D9. The method of embodiment D1, wherein said host is a mammal.
   D10. The method of embodiment D9, wherein said mammal is a human.
   D11. The method of any of embodiments D1-D10, wherein said cells are contacted with said recombinant plasmid vector at the same time, at an earlier time, or at a later time, as the contacting of said cells with a different recombinant plasmid vector.
   D12. The method of embodiment D5, wherein said administration of said recombinant plasmid vector to said animal is at the same time, at an earlier time, or at a later time as the administration of a different recombinant plasmid vector to said animal.
   D13. The method of embodiment D1, wherein said injection further comprises vesicular stomatitis virus vector.
   D14. The method of embodiment D5, wherein said recombinant plasmid vector is administered with another DNA vaccine, or virus-vector based vaccine.
   E1. The method of any of embodiments A1-A28, B1-B38, C1-C34, or D1-D14, wherein said vesicular stomatitis virus vector encodes a heterologous protein or a modified vesicular stomatitis virus surface protein that reduces immunity to vesicular stomatitis virus vector on subsequent deliveries of a vesicular stomatitis virus vector.
   E3. The method of any of embodiments A1-A28, B1-B38, C1-C34, or D1-D14, wherein said vesicular stomatitis virus vector encodes a heterologous protein or a modified vesicular stomatitis virus surface protein that causes targeting of said virus vector to receptors on a specific cell type.
   E5. The method of any of embodiments A1-A28, B1-B38, C1-C34, or D1-D14, wherein said vesicular stomatitis virus vector is propagation defective.
   E6. The method of embodiment E5, wherein the M and G genes of the virus vector are deleted or mutated such that the virus vector particle is replication-deficient.
   E7. The method of embodiment E5, wherein said virus vector is present in a virus vector particle, and said virus vector particle is produced by the method comprising
(a) transfecting a permissive producer cell with a vector comprising a nucleic acid sequence of at least part of the VSV genome and T7 RNA polymerase wherein the M and G genes are deleted;
(b) growing said producer cell under cell culture conditions sufficient to allow producing of vesicular stomatitis virus vector particles in said cell;
(c) co-transfecting said cell with plasmids encoding M and G genes; and
(d) collecting said particles.
   E8. The method of embodiment E7, wherein said producer cell is grown in cell culture medium, and wherein said replication-defective vector particles are collected from said medium.
   E9. The method of embodiment E7, wherein said replication-defective vector particles are collected from said producer cells.
   F1. A VSV expression system, comprising
a) a recombinant plasmid vector comprising a polynucleotide comprising the following elements operably linked 5′ to 3′: (i) a bacteriophage promoter sequence, and (ii) at heterologous gene; and
b) a vesicular stomatitis virus vector particle (VSV) comprising a polynucleotide encoding a bacteriophage RNA polymerase that operates on said bacteriophage promoter sequence.
   F2. The system of embodiment F1, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   F3. The system of any of embodiments F1-F2, wherein said plasmid vector is circular.
   F4. The system of any of embodiments F1-F2, wherein said plasmid vector is linear.
   F5. The system of any of embodiments F1-F3, wherein said plasmid comprises chemically modified DNA.
   F6. The system of any of embodiments F1-F5, wherein said heterologous gene comprises a sequence encoding an internal ribosome entry site.
   F7. The system of any of embodiments F1-F6, further comprising a DNA sequence encoding a vaccinia capping enzyme.
   F8. The system of embodiments F1-F6, further comprising a first DNA sequence encoding the D1 catalytic subunit of vaccinia capping enzyme, and a second DNA sequence encoding the D12 subunit of vaccinia capping enzyme.
   F9. The system of embodiment F8, comprising a recombinant plasmid capping vector comprising said first DNA sequence and said second DNA sequence.
   F10. The system of embodiment F8, comprising a first recombinant plasmid capping vector comprising said first DNA sequence and with a second recombinant plasmid capping vector comprising said second DNA sequence.
   F11. The system of any of embodiments F8-F10, wherein said first DNA sequence comprises the sequence set forth in SEQ ID NO. 5 and said second DNA sequence comprises the sequence set forth in SEQ ID NO. 6.
   F12. The system of any of embodiments F1-F11, wherein said bacteriophage promoter sequence is operably linked to a bacteriophage gene expression regulator sequence.
   F13. The system of embodiment F12, wherein said regulator sequence is selected from the group consisting of a ribo-switch sequence and a ligand-regulated protein binding site.
   F14. The system of any of embodiments F1-F13, further comprising a DNA sequence encoding a protein that regulates bacteriophage RNA polymerase activity.
   F15. The system of embodiment F14, wherein said DNA sequence encodes a lysozyme.
   F16. The system of embodiment F14 or F15, wherein the recombinant plasmid vector of embodiment F1 comprises said DNA sequence encoding the protein that regulates bacteriophage RNA polymerase activity.
   F17. The system of embodiment F14 or F15, comprising a recombinant plasmid RNA polymerase regulator vector comprising said DNA sequence encoding the protein that regulates bacteriophage RNA polymerase activity.
   F18. The system of any of embodiments F1-F17, further comprising a DNA sequence encoding a protein that modulates the immune response of a host animal.
   F19. The system of embodiment F18, wherein said host animal is a human.
   F20. The system of embodiment F18, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   F21. The system of embodiment F20, wherein said enhancer of antigen presentation to APCs is GM-CSF.
   F22. The system of embodiment F20, wherein said factor that helps recruit and activate DCs is MIP-1α or Flt3L.
   F23. The system of embodiment F20, wherein said enhancer of T-lymphocyte priming is B7-2 or CD154.
   F24. The system of embodiment F20, wherein said stimulator of T-lymphocyte expansion is selected from the group consisting of IL-2, IL-12, and IL-15.
   F25. The system of embodiment F18, wherein the recombinant plasmid vector of embodiment F1 comprises said DNA sequence encoding the protein that modulates the immune response of a host animal.
   F26. The system of any of embodiments A1-A17, further comprising a recombinant plasmid immune modulating vector comprising said DNA sequence encoding the protein that modulates the immune response of a host animal.
   F27. The system of any of embodiments F1-F26, wherein said recombinant plasmid vector comprises a T7 promoter corresponding to residues 794 to 813 of SEQ ID No. 3.
   F28. The system of any of embodiments F1-F27, wherein said vesicular stomatitis virus vector particle comprises the polynucleotide sequence of SEQ ID No. 1.
   G1. A system for producing two or more heterologous proteins in a cell, comprising
a) a recombinant plasmid vector comprising a polynucleotide comprising the following elements operably linked 5′ to 3′: (i) a bacteriophage promoter sequence, and (ii) a DNA sequence encoding two or more heterologous proteins; and
b) a vesicular stomatitis virus vector particle (VSV) comprising a polynucleotide encoding a bacteriophage RNA polymerase that operates on said bacteriophage promoter sequence.
   G2. The system of embodiment G1, wherein said DNA sequence encoding two or more heterologous proteins comprises two or more sequences encoding internal ribosome entry sites, each of which enables translation of a different heterologous protein.
   G3. The system of embodiment G1, wherein said heterologous gene encodes a fusion protein comprising two or more heterologous proteins.
   G4. The system of embodiment G1, wherein said plasmid comprises two or more bacteriophage promoter sequences, and said system further comprises a DNA sequence encoding a DNA endonuclease, capable of expression in a cell and releasing individual expression cassettes from said plasmid, said expression cassettes comprising a bacteriophage promoter sequence and a DNA sequence encoding a heterologous protein.
   G5. The system of embodiment G4, wherein said recombinant plasmid comprises said DNA sequence encoding a DNA endonuclease.
   G6. The system of embodiment G5, comprising a second recombinant plasmid vector, wherein said second recombinant plasmid vector comprises a DNA sequence encoding a DNA endonuclease.
   G7. The system of embodiment G1, further comprising a DNA sequence encoding a ribozyme, said ribozyme is capable of expression in a cell, and cleaving transcripts encoding said two or more heterologous proteins to provide two or more individual transcripts, each of which codes for a different heterologous protein.
   G8. The system of embodiment G7, wherein each of said individual transcripts comprises an internal ribosome entry sequence.
   G9. The system of any of embodiments G1-G8, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   G10. The system of any of embodiments G1-G9, wherein said plasmid vector is circular.
   G11. The system of any of embodiments G1-G9, wherein said plasmid vector is linear.
   G12. The system of any of embodiments G1-G11, wherein said plasmid comprises chemically modified DNA.
   G13. The system of any of embodiments G1-G11, wherein said plasmid vector comprises a DNA sequence encoding three or more heterologous proteins.
   G14. The system of any of embodiments G1-G11, wherein said plasmid vector comprises a DNA sequence encoding four or more heterologous proteins.
   015. The system of any of embodiments G1-G11, wherein said plasmid vector comprises a DNA sequence encoding five or more heterologous proteins.
   G16. The system of any of embodiments G1-G15, further comprising a DNA sequence encoding a vaccinia capping enzyme.
   G17. The system of any of embodiments G1-G15, further comprising a first DNA sequence encoding the D1 catalytic subunit of vaccinia capping enzyme, and a second DNA sequence encoding the D12 subunit of vaccinia capping enzyme.
   G18. The system of embodiment G17, comprising a recombinant plasmid capping vector comprising said first DNA sequence and said second DNA sequence.
   G19. The system of embodiment G17, comprising a first recombinant plasmid capping vector comprising said first DNA sequence and with a second recombinant plasmid capping vector comprising said second DNA sequence.
   G20. The system of any of embodiments G17-G19, wherein said first DNA sequence comprises the sequence set forth in SEQ ID NO. 5 and said second DNA sequence comprises the sequence set forth in SEQ ID NO. 6.
   G21. The system of any of embodiments G1-G20, wherein said bacteriophage promoter sequence is operably linked to a bacteriophage gene expression regulator sequence.
   G23. The system of embodiment G21, wherein said regulator sequence is selected from the group consisting of a ribo-switch sequence and a ligand-regulated protein binding site.
   G24. The system of any of embodiments G1-G23, further comprising a DNA sequence encoding a protein that regulates bacteriophage RNA polymerase activity.
   G25. The system of embodiment G24, wherein said DNA sequence encodes a lysozyme.
   G26. The system of embodiment G24 or G25, wherein the recombinant plasmid vector of embodiment G1 comprises said DNA sequence encoding the protein that regulates bacteriophage RNA polymerase activity.
   G27. The system of embodiment G24 or G25, comprising a recombinant plasmid RNA polymerase regulator vector comprising said DNA sequence encoding the protein that regulates bacteriophage RNA polymerase activity.
   G28. The system of any of embodiments G1-G27, further comprising a DNA sequence encoding a protein that modulates the immune response of a host animal.
   G29. The system of embodiment G28, wherein said host animal is a human.
   G30. The system of embodiment G28, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   G31. The system of embodiment G30, wherein said enhancer of antigen presentation to APCs is GM-CSF.
   G32. The system of embodiment G30, wherein said factor that helps recruit and activate DCs is MIP-1α or Flt3L.
   G33. The system of embodiment G30, wherein said enhancer of T-lymphocyte priming is B7-2 or CD154.
   G34. The system of embodiment G30, wherein said stimulator of T-lymphocyte expansion is selected from the group consisting of IL-2, IL-12, and IL-15.
   G35. The system of embodiment G28, wherein the recombinant plasmid vector of embodiment G1 comprises said DNA sequence encoding the protein that modulates the immune response of a host animal.
   G36. The system of embodiment G28, comprising a recombinant plasmid immune modulating vector comprising said DNA sequence encoding the protein that modulates the immune response of a host animal.
   G37. The system of any of embodiments G1-G36, wherein said recombinant plasmid vector comprises a T7 promoter corresponding to residues 794 to 813 of SEQ ID No. 3.
   G38. The system of any of embodiments G1-G37, wherein said vesicular stomatitis virus vector particle comprises the polynucleotide sequence of SEQ ID No. 1.
   H1. A system for producing two or more heterologous proteins in a cell, comprising
a) two or more recombinant plasmid vectors, each comprising a polynucleotide comprising the following elements operably linked 5′ to 3′: (i) a bacteriophage promoter sequence, and (ii) a DNA sequence encoding a heterologous protein; and
b) a vesicular stomatitis virus vector particle (VSV) comprising a polynucleotide encoding a bacteriophage RNA polymerase that operates on said bacteriophage promoter sequence.
   H2. The system of embodiment H1, wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters.
   H3. The system of any of embodiments H1-H2, wherein said plasmid vectors are circular.
   H4. The system of any of embodiments H1-H2, wherein said plasmid vectors are linear.
   H5. The system of any of embodiments H1-H3, wherein said plasmid vectors comprise chemically modified DNA.
   H6. The system of any of embodiments H1-H5, wherein said heterologous genes comprise a sequence encoding an internal ribosome entry site.
   H7. The system of any of embodiments H1-H6, comprising three or more recombinant plasmid vectors, each comprising a DNA sequence encoding a heterologous protein.
   H8. The system of any of embodiments H1-H6, comprising four or more recombinant plasmid vectors, each comprising a DNA sequence encoding a heterologous protein.
   H9. The system of any of embodiments H1-H6, comprising five or more recombinant plasmid vectors, each comprising a DNA sequence encoding a heterologous protein.
   H10. The system of any of embodiments H1-H6, comprising six or more recombinant plasmid vectors, each comprising a DNA sequence encoding a heterologous protein.
   H11. The system of any of embodiments H1-H6, comprising seven or more recombinant plasmid vectors, each comprising a DNA sequence encoding a heterologous protein.
   H12. The system of any of embodiments H1-H6, comprising eight or more recombinant plasmid vectors, each comprising a DNA sequence encoding a heterologous protein.
   H13. The system of any of embodiments H1-H12, further comprising a DNA sequence encoding a vaccinia capping enzyme.
   H14. The system of any of embodiments H1-H12, further comprising a first DNA sequence encoding the D1 catalytic subunit of vaccinia capping enzyme, and a second DNA sequence encoding the D12 subunit of vaccinia capping enzyme.
   H15. The system of embodiment H14, comprising a recombinant plasmid capping vector comprising said first DNA sequence and said second DNA sequence.
   H16. The system of embodiment H14, comprising a first recombinant plasmid capping vector comprising said first DNA sequence and with a second recombinant plasmid capping vector comprising said second DNA sequence.
   H17. The system of any of embodiments H14-H16, wherein said first DNA sequence comprises the sequence set forth in SEQ ID NO. 5 and said second DNA sequence comprises the sequence set forth in SEQ ID NO. 6.
   H18. The system of any of embodiments H1-H17, wherein said bacteriophage promoter sequence is operably linked to a bacteriophage gene expression regulator sequence.
   H19. The system of embodiment H18, wherein said regulator sequence is selected from the group consisting of a ribo-switch sequence and a ligand-regulated protein binding site.
   H20. The system of any of embodiments H1-H19, further comprising a DNA sequence encoding a protein that regulates bacteriophage RNA polymerase activity.
   H21. The system of embodiment H₂O, wherein said DNA sequence encodes a lysozyme.
   H22. The system of embodiment H20 or H21, wherein at least one recombinant plasmid vector of embodiment H1 comprises said DNA sequence encoding the protein that regulates bacteriophage RNA polymerase activity.
   H23. The system of embodiment H20 or H21, comprising a recombinant plasmid RNA polymerase regulator vector comprising said DNA sequence encoding the protein that regulates bacteriophage RNA polymerase activity.
   H24. The system of any of embodiments H1-H23, further comprising DNA sequence encoding a protein that modulates the immune response of a host animal.
   H25. The system of embodiment H24, wherein said host animal is a human.
   H26. The system of embodiment H24, wherein said DNA sequence encodes a protein selected from the group consisting of an enhancer of antigen presentation to APCs, a factor that helps recruit and activate DCs, an enhancer of T-lymphocyte priming, and a stimulator of T-lymphocyte expansion.
   H27. The system of embodiment H26, wherein said enhancer of antigen presentation to APCs is GM-CSF.
   H28. The system of embodiment H26, wherein said factor that helps recruit and activate DCs is MIP-1α or Flt3L.
   H29. The system of embodiment H26, wherein said enhancer of T-lymphocyte priming is B7-2 or CD154.
   H30. The system of embodiment H26, wherein said stimulator of T-lymphocyte expansion is selected from the group consisting of IL-2, IL-12, and IL-15.
   H31. The system of embodiment H24, wherein at least one recombinant plasmid vector of embodiment H1 comprises said DNA sequence encoding the protein that modulates the immune response of a host animal.
   H32. The system of embodiment H24, comprising a recombinant plasmid immune modulating vector comprising said DNA sequence encoding the protein that modulates the immune response of a host animal.
   H33. The system of any of embodiments H1-H32, wherein at least one of said recombinant plasmid vectors comprises a T7 promoter corresponding to residues 794 to 813 of SEQ ID No. 3.
   H34. The system of any of embodiments H1-H33, wherein said vesicular stomatitis virus vector particle comprises the polynucleotide sequence of SEQ ID No. 1.
   I1. The system of any of embodiments F1-F28, G1-G38, or H1-H34, formulated for injection into a host.
   I2. The system of any of embodiments F1-F28, G1-G38, or H1-H34, further comprising an agent that promotes entry of DNA into cells.
   I3. The system of embodiment I3, wherein said agent is selected from the group consisting of lipids, polymers, and gold particles.
   I4. The system of any of embodiments F1-F28, G1-G38, or H1-H34, formulated for transfecting cells by a method selected from the group consisting of liposome mediated transfer, lipofection, polycation-mediated transfer, direct DNA transfer, electroporation, gene gun use, transfection in the presence of polymers, and transfection in the presence of gold particles.
   I5. The system of any of embodiments F1-F28, G1-G38, or H1-H34, formulated for administration to an animal.
   I6. The system of embodiment I5, wherein said animal is a mammal.
   I7. The system of embodiment I6, wherein said mammal is a human.
   I8. The system of embodiment 15, wherein said administration is by a method selected from the group consisting of intra-muscular injection, intra-peritoneal injection, intravenous delivery, subcutaneous deliver, intra-nasal delivery, and oral delivery.
   I9. The system of embodiment I1, wherein said host is a mammal.
   I10. The system of embodiment I9, wherein said mammal is a human.
   J1. The system of any of embodiments F1-F28, G1-G38, H1-H34, or I1-I14, wherein said vesicular stomatitis virus vector encodes a heterologous protein or a modified vesicular stomatitis virus surface protein that reduces immunity to vesicular stomatitis virus vector on subsequent deliveries of a vesicular stomatitis virus vector.
   J3. The system of any of embodiments F1-F28, G1-G38, H1-H34, or I1-I14, wherein said vesicular stomatitis virus vector encodes a heterologous protein or a modified vesicular stomatitis virus surface protein that causes targeting of said virus vector to receptors on a specific cell type.
   J5. The system of any of embodiments F1-F28, G1-G38, H1-H34, or I1-I14, wherein said vesicular stomatitis virus vector is propagation defective.
   J6. The system of embodiment J5, wherein the M and G genes of the virus vector are deleted or mutated such that the virus vector particle is replication-deficient.
   J7. The system of embodiment J5, wherein said virus vector is present in a virus vector particle, and said virus vector particle is produced by the method comprising
(e) transfecting a permissive producer cell with a vector comprising a nucleic acid sequence of at least part of the VSV genome and T7 RNA polymerase wherein the M and G genes are deleted;
(f) growing said producer cell under cell culture conditions sufficient to allow producing of vesicular stomatitis virus vector particles in said cell;
(g) co-transfecting said cell with plasmids encoding M and G genes; and
(h) collecting said particles.
   J8. The system of embodiment J7, wherein said producer cell is grown in cell culture medium, and wherein said replication-defective vector particles are collected from said medium.
   J9. The system of embodiment J7, wherein said replication-defective vector particles are collected from said producer cells.

The entirety of each patent, patent application, publication, document and sequence (e.g., nucleotide sequence, amino acid sequence) referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1. A method for expressing a heterologous protein in a cell, comprising contacting said cell with
(i)a. a recombinant plasmid vector comprising a polynucleotide comprising the following elements operably linked 5′ to 3′: (i) a bacteriophage promoter sequence, and (ii) a heterologous gene; and
b. a vesicular stomatitis virus vector particle (VSV) comprising a polynucleotide encoding a bacteriophage RNA polymerase that operates on said bacteriophage promoter sequence;
(ii) the method of (i), wherein said bacteriophage RNA polymerase is selected from the group consisting of T7, SP6, T1, T3, and T5 RNA polymerases and said bacteriophage RNA polymerase promoter is selected from the group consisting of T7, SP6, T1, T3 and T5 promoters;
(iii) the method of (i) or (ii), wherein said plasmid vector is circular, is linear, or comprises a chemically modified DNA.
(iv) the method of (iii), wherein said heterologous gene comprises a sequence encoding an internal ribosome entry site;
(v) the method of (i), further comprising contacting said cell with a DNA sequence encoding a vaccinia capping enzyme; or further comprising contacting said cell with a first DNA sequence encoding the D1 catalytic subunit of vaccinia capping enzyme, and a second DNA sequence encoding the D12 subunit of vaccinia capping enzyme; or further comprising contacting said cell with a recombinant plasmid capping vector comprising said first DNA sequence and said second DNA sequence; or further comprising contacting said cell with a first recombinant plasmid capping vector comprising said first DNA sequence and with a second recombinant plasmid capping vector comprising said second DNA sequence;
(vi) the method of any of (v), wherein said first DNA sequence comprises the sequence set forth in SEQ ID NO:5 and said second DNA sequence comprises the sequence set forth in SEQ ID NO:6;
(vii) the method of (i), wherein said bacteriophage promoter sequence is operably linked to a bacteriophage gene expression regulator sequence or a regulator sequence is selected from the group consisting of a ribo-switch sequence and a ligand-regulated protein binding site
(viii) the method of (i), further comprising contacting said cell with a DNA sequence encoding a protein that regulates bacteriophage RNA polymerase activity, or contacting said cell with a DNA sequence encoding a lysozyme, or contacting said cell with a DNA sequence encoding a protein that regulates a bacteriophage RNA polymerase activity; or contacting said cell with a recombinant plasmid RNA polymerase regulator vector comprising said DNA sequence encoding the protein that regulates bacteriophage RNA polymerase activity;
(ix) the method of (i), wherein said immune response modulator modulates the immune response of a host animal, or a human;
(x) the method of (ix), wherein said immune response modulator is selected from the group consisting of an enhancer of antigen presentation to APCs, a factor that helps recruit and activate DCs, an enhancer of T-lymphocyte priming, and a stimulator of T-lymphocyte expansion;
(xi) the method of (x), wherein said enhancer of antigen presentation to APCs is GM-CSF, or said factor that helps recruit and activate DCs is MIP-I OC or Flt3L, or said enhancer of T-lymphocyte priming is B7-2 or CD154, or said stimulator of T-lymphocyte expansion is selected from the group consisting of IL-2, IL-12, and IL-15;
(xii) the method of (i), wherein said recombinant plasmid vector comprises a T7 promoter corresponding to residues 794 to 813 of SEQ ID No. 3;
(xiii) the method of (i), wherein said vesicular stomatitis virus vector particle comprises the polynucleotide sequence of SEQ ID NO:1; or
(xiv) the method of (i), wherein said heterologous gene codes for an immune response modulator.

2-27. (canceled)

28. A method for producing two or more heterologous proteins in a cell, comprising contacting said cell with
(i)a. a recombinant plasmid vector comprising a polynucleotide comprising the following elements operably linked 5′ to 3′: (i) a bacteriophage promoter sequence, and (ii) a DNA sequence encoding two or more heterologous proteins; and
b. a vesicular stomatitis virus vector particle (VSV) comprising a polynucleotide encoding a bacteriophage RNA polymerase that operates on said bacteriophage promoter sequence;
(ii) the method of (i), wherein said DNA sequence encoding two or more heterologous proteins comprises two or more sequences encoding internal ribosome entry sites, each of which enables translation of a different heterologous protein, or said DNA sequence encodes a fusion protein comprising two or more heterologous proteins;
(iii) the method of (i), wherein said plasmid comprises two or more bacteriophage promoter sequences, and said cell is contacted with a DNA sequence encoding a DNA endonuclease, said DNA endonuclease is expressed in said cell, and said DNA endonuclease releases individual expression cassettes from said plasmid, said expression cassettes comprising a bacteriophage promoter sequence and a DNA sequence encoding a heterologous protein;
(iv) the method of (i), wherein said recombinant plasmid comprises said DNA sequence encoding a DNA endonuclease;
(v) the method of (i), further comprising contacting said cell with a second recombinant plasmid vector, wherein said second recombinant plasmid vector comprises a DNA sequence encoding a DNA endonuclease; or further comprising contacting said cell with a DNA sequence encoding a ribozyme, said ribozyme is expressed in said cell, wherein said ribozyme cleaves transcripts encoding said two or more heterologous proteins to provide two or more individual transcripts, each of which codes for a different heterologous protein;
(vi) the method of (V), wherein each of said individual transcripts comprises an internal ribosome entry sequence; or
(vii) the method of (i), wherein said plasmid vector is circular, or is linear, or comprises a chemically modified DNA.

29-88. (canceled)

89. A VSV expression system, comprising
(i)a. a recombinant plasmid vector comprising a polynucleotide comprising the following elements operably linked 5′ to 3′: (i) a bacteriophage promoter sequence, and (ii) at heterologous gene; and
b. a vesicular stomatitis virus vector particle (VSV) comprising a polynucleotide encoding a bacteriophage RNA polymerase that operates on said bacteriophage promoter sequence; or
(ii) the VSV expression system of (i), wherein at least one of said heterologous proteins modulates the immune response of an animal.

90-100. (canceled)
